(19) United States
(12) Patent Application Publication
(10) Pub. No.: US 2024/0190978 A1
(43) Pub. Date: Jun. 13, 2024

(54) COMPOSITIONS AND METHODS FOR IMMUNOMODULATORY BIFUNCTIONAL FUSION MOLECULES

(71) Applicant: CSBioAsset LLC, Claymont, DE (US)

(72) Inventor: Chao SHI, Claymont, DE (US)

(21) Appl. No.: 18/509,203

(22) Filed: Nov. 14, 2023

Related U.S. Application Data
(60) Provisional application No. 63/383,852, filed on Nov. 15, 2022.

(51) Int. Cl. C07K 16/28; A61K 39/00; A61K 39/39; A61P 37/06; C07K 14/705

(52) U.S. Cl. CPC C07K 16/2866; A61K 39/39; A61P 37/06; C07K 14/70521; C07K 16/2827; C07K 16/2878; A61K 2039/55516; C07K 2317/31; C07K 2319/30

(57) ABSTRACT

The disclosure is directed to a bispecific protein, comprising a first binding domain (BD1) that binds to a first target and a second binding domain (BD2) that binds to a second target, wherein the BD1 binds to IL6R or CD40, and BD2 binds to CD80/CD86; a composition comprising the bispecific protein, nucleic acids, cells, or any methods of use thereof.

### CROSS-REFERENCE TO RELATED APPLICATIONS

This application claims the benefit of U.S. Provisional Application No. 63/383,852, filed Nov. 15, 2022, which is hereby incorporated by reference in its entirety.

### CROSS-REFERENCE TO ELECTRONICALLY SUBMITTED SEQUENCE LISTING

The content of the electronically submitted sequence listing in .XML file (Name: 5122_0010002_SequenceListing_ST26; Size: 101,683 bytes; and Date of Creation: Nov. 13, 2023) filed with the application is incorporated herein by reference in its entirety.

### FIELD OF DISCLOSURE

The present disclosure provides bispecific proteins that bind two targets (e.g., molecules, epitopes, or cellular receptors) and that are bivalent for binding to each of the first and second targets.

### BACKGROUND OF DISCLOSURE

Immunological disorders are diseases or conditions caused by a dysfunction of the immune system and include allergy, asthma, autoimmune diseases, autoinflammatory syndromes and immunological deficiency syndromes. Most immunological disorders exhibit clinical heterogeneity, with a polygenic nature and multifactorial contributions from both genetic factors and environmental factors.

Traditional therapies for autoimmune disease and allergic disease have relied on immunosuppressive medications such as steroids or cytotoxic agents that globally dampen immune responses. These agents are effective for many patients. However, long-term treatments with high doses are often needed to maintain disease control, leaving the patient susceptible to life-threatening opportunistic infections and long-term risk of malignancy. Therefore, treatments that specifically target the components of the immune system involved in the diseases are preferred.

Newer treatment options, including biologics, have been developed to target innate immunity, B cells, T cells, and cytokines. While these agents have improved clinical profile versus steroids and other blunt immunosuppressive medications, there is a significant inter-individual variability of response to them. Only 30% patients achieve a remission after treatment, and 20% of them do not respond anymore after 24 weeks. Although the exact cause for the variability is unknown, it has been hypothesized that given the polygenic nature of diseases, a targeted therapy might be less effective in some circumstances because it only covers a part of the pathogenic pathways.

One solution to the problem is to treat patients simultaneously with two or more such therapeutic agents. The idea has been tested in rheumatoid arthritis with a combination of two biologics, specifically anti-TNF and anti-IL1 agents, but results are disappointing showing increased side effects but no improved efficacy. It is clear that simply putting two drugs together will not automatically achieve synergistic or additive efficacy in these circumstances. Another solution used in practice is to treat patients sequentially with agents targeting different pathways. Patients are initially prescribed a medication to try. If the treatment does not result in a response or start losing efficacy after a period of time, another medication will be prescribed. Consequently, patients often rotate through multiple treatment options after being treated for years. This not only creates significant financial and treatment burdens, but also delays the timing for receiving the optimal treatment. Therefore, a more effective, safer, and efficient approach for treating immunological disorders is needed.

### SUMMARY OF THE DISCLOSURE

The disclosure provides bispecific proteins that bind two targets (e.g., molecules, epitopes, or cellular receptors) and that are bivalent for binding to each of the first and second targets. The disclosure also provides methods of modulating immune responses in a subject as well as methods for treating immunological disorders (e.g., diseases or conditions caused by a dysfunction of the immune system, including allergy, asthma, autoimmune diseases, autoinflammatory syndromes and immunological deficiency syndromes) or cancer in a subject (e.g., a human subject) by administering the proteins, nucleic acid molecules and/or compositions to the subject.

The present disclose includes a bispecific protein, comprising a first binding domain (BD1) that binds to a first target and a second binding domain (BD2) that binds to a second target (i) wherein the BD1 binds to IL6R, and BD2 binds to CD80/CD86; or (ii) wherein the BD1 binds to CD40 and BD2 binds to CD80/CD86. In some aspects, the BD1 binds to IL6R, and BD2 binds to CD80/CD86, and the bispecific protein has antagonistic effects on the signaling of IL6 and T cell activation. In some aspects, the BD1 binds to CD40, and the BD2 binds to CD80/CD86; and the bispecific protein has antagonistic effects on the signaling of CD40 and T cell activation. In some aspects, the bispecific protein further comprises an Fc region.

In some aspects, the BD1 in the bispecific protein comprises a Fab domain, a single-chain variable fragment (scFv), a single domain antibody, an antibody variable domain, or any combination thereof. In some aspects, the BD1 is connected to the Fc region via an antibody hinge region. In some aspects, the BD2 in the bispecific protein comprises a scFv or one or multiple fragments of a cellular receptor. In some aspects, the BD2 is connected to the Fc region via a polypeptide linker.

In some aspects, the linker for the bispecific protein comprises a GS linker. In some aspects, the linker comprises an amino acid sequence of the formula (GGGGS)n or (GGGGS)ₙG, wherein n represents the number of repeating GGGGS units and is a positive integer (e.g., 1, 2, 3, 4, 5, 6, 7, 8, 9 or 10). In some aspects, the Fc region comprises a domain selected from the group consisting of an Fc region from an IgG1, IgG2, IgG3, IgG4, IgA, lgM, lgE, and lgD. In some aspects, the Fc region is modified with various glycosylation patterns or aglycosylated. In some aspects, the Fc region comprises an IgG1 constant region comprising one or more amino acid substitutions selected from the group consisting of D265A, N297A, K322A, L234F, L235E and P331S. In some aspects, the Fc region comprises an IgG4 constant region comprising a S228P mutation.

In some aspects, the bispecific protein comprises (i) a first polypeptide chain comprising, from N-terminus to C-terminus VH-CH1-CH2-CH3-L1-BD2; VH-CH1-CH2-CH3(N-term)-L1-BD2-L2-CH3(C-term); VH-CH1-CH2-L1-BD2-L2-CH3; or VH-CH1-CH2 (N-term)-L1-BD2-L2-CH2(C-term)-CH3; and (ii) a second polypeptide chain comprising VL-CL1, wherein the VH and VL binds to IL6R or CD40, wherein L1 and L2 are optional linkers. In some aspects, the BD2 comprises a scFv. In some aspects, the BD2 comprises one or more ligand binding domain of a cellular receptor.

In some aspects, the bispecific protein of the present disclosure comprises two identical first polypeptide chains and two identical second polypeptide chains. In some aspects, the bispecific protein comprises two first polypeptide chains (PPT-1 and PPT-2) that are different and/or two second polypeptide chains that are different. In some aspects, the PPT-1 comprises a CH2-CH3 domain comprising T366W/S354C substitutions and the PPT-2 comprises a CH2-CH3 domain comprising T366S/L368A/Y407V/Y349C substitutions.

In some aspects, the BD2 in the bispecific protein comprises an ectodomain of CTLA4. In some aspects, the ectodomain of CTLA-4 comprises an amino acid sequence having at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to SEQ ID Nos: 35 to 40, wherein the amino acid sequence is capable of binding to CD80/CD86. In some aspects, the ectodomain of CTLA-4 comprises an amino acid sequence comprising SEQ ID NO: 41 (MYPPPY), wherein the amino acid sequence is capable of binding to CD80 and CD86.

In some aspects, the BD1 in the bispecific protein comprises a heavy chain immunoglobulin variable domain (VH) and a light chain immunoglobulin variable domain (VL) that bind to IL6R, wherein:
(a) the VH comprises a VH-CDR1 sequence of SDHAWS (SEQ ID NO: 19), a VH-CDR2 sequence of YISYSGITTYNPSLKS (SEQ ID NO: 20), and a VH-CDR3 sequence of SLARTTAMDY (SEQ ID NO: 21), and
(b) the VL comprises a VL-CDR1 sequence of RASQDISSYLN (SEQ ID NO: 22), a VL-CDR2 sequence of YTSRLHS (SEQ ID NO: 23), and a VL-CDR3 sequence of QQGNTLPYT (SEQ ID NO: 24).

In some aspects, the VH in the bispecific protein comprises an amino acid sequence that has at least about 80%, at least about 85%, at least about 95%, at least about 96%, at least about 97%, at least about 98%, at least about 99%, or about 100% sequence identity to SEQ ID NO: 25; and/or the VL comprises an amino acid sequence that has at least about 80%, at least about 85%, at least about 95%, at least about 96%, at least about 97%, at least about 98%, at least about 99%, or about 100% sequence identity to SEQ ID NO: 26.

In some aspects, the bispecific protein comprises a heavy chain (HC) selected from the group consisting of the amino acid sequence as set forth in SEQ ID NOs: 3 to 10, or a variant thereof having one or more conservative amino acid substitutions, and a light chain (LC) comprising the amino acid sequence as set forth SEQ ID NO: 1, or a variant thereof having one or more conservative amino acid substitutions.

In some aspects, the heavy chain in the bispecific protein comprises an amino acid sequence having at least about 95% sequence identity to the HC sequence as set forth in any one of SEQ ID NOs: 3 to 10; and/or the light chain comprises an amino acid sequence having at least 95% sequence identity to the LC sequence as set forth in SEQ ID NO: 1.

In some aspects, the BD1 in the bispecific protein comprises a heavy chain immunoglobulin variable domain (VH) and a light chain immunoglobulin variable domain (VL) derived from the following anti-IL6R antibodies: tocilizumab (SEQ ID NO: 42 and SEQ ID NO: 43), sarilumab (SEQ ID NO: 44 and SEQ ID NO: 45), satralizumab (SEQ ID NO: 46 and SEQ ID NO: 47), olokizumab (SEQ ID NO: 48 and SEQ ID NO: 49), and vobarilizumab (SEQ ID NO: 50). In some aspects, the heavy chain and/or the light chain in the bispecific protein comprises an amino acid sequence having at least about 80%, at least about 85%, at least about 95%, at least about 96%, at least about 97%, at least about 98%, at least about 99%, or about 100% sequence identity to the sequence as set forth in any one of SEQ ID NOs: 42 to 50.

In some aspects, the BD1 in the bispecific protein comprises a heavy chain immunoglobulin variable domain (VH) and a light chain immunoglobulin variable domain (VL), which bind to CD40, wherein:
(a) the VH comprises a VH-CDR1 sequence of GFTFSSYGMH (SEQ ID NO: 27), a VH-CDR2 sequence of VISYEESNRYHADSVKG (SEQ ID NO: 28), and a VH-CDR3 sequence of DGGIAAPGPDY (SEQ ID NO: 29), and
(b) the VL comprises a VL-CDR1 sequence of RSSQSLLYSNGYNYLD (SEQ ID NO: 30), a VL-CDR2 sequence of LGSNRAS (SEQ ID NO: 31), and a VL-CDR3 sequence of MQARQTPF (SEQ ID NO: 32).

In some aspects, the VH in the bispecific protein comprises an amino acid sequence that has at least about 80%, at least about 85%, at least about 95%, at least about 96%, at least about 97%, at least about 98%, at least about 99%, or about 100% sequence identity to SEQ ID NO: 33; and/or the VL comprises an amino acid sequence that has at least about 80%, at least about 85%, at least about 95%, at least about 96%, at least about 97%, at least about 98%, at least about 99%, or about 100% sequence identity to SEQ ID NO: 34.

In some aspects, the bispecific protein comprises a heavy chain selected from the group consisting of the amino acid sequence as set forth in SEQ ID Nos: 11 to 18, and a light chain comprising the amino acid sequence as set forth in SEQ ID No: 2. In some aspects, the heavy chain comprises an amino acid sequence having at least about 95% sequence identity to the HC sequence as set forth in SEQ ID NOs: 11 to 18; and/or the light chain comprises an amino acid sequence having at least about 95% sequence identity to the LC sequence as set forth in SEQ ID NO: 2.

In some aspects, the BD1 in the bispecific protein comprises a heavy chain immunoglobulin variable domain (VH) and a light chain immunoglobulin variable domain (VL) derived from the following anti-CD40 antibodies: iscalimab (SEQ ID NO: 51 and SEQ ID NO: 52), bleselumab (SEQ ID NO: 53 and SEQ ID NO: 54), ravagalimab (SEQ ID NO: 55 and SEQ ID NO: 56), lucatumumab (SEQ ID NO: 57 and SEQ ID NO: 58), BMS-986325 (SEQ ID NO: 59 and SEQ ID NO: 60), teneliximab (SEQ ID NO: 61 and SEQ ID NO: 62), BI-655064 (SEQ ID NO: 63 and SEQ ID NO: 64), and KPL-404 (SEQ ID NO: 65 and SEQ ID NO: 66). In some aspects, the heavy chain and/or the light chain comprises an amino acid sequence having at least about 80%, at least about 85%, at least about 95%, at least about 96%, at least about 97%, at least about 98%, at least about 99%, or about 100% sequence identity to the sequence as set forth in any one of SEQ ID NOs: 51 to 66.

In some aspects, the bispecific protein further comprises a non-polypeptide moiety. In some aspects, the bispecific protein further comprises a heterologous moiety fused to the BD1 and/or the BD2. In some aspects, the heterologous moiety is a half-life extending moiety. In some aspects, the heterologous moiety comprises a non-polypeptide moiety. In some aspects, the heterologous moiety comprises a polypeptide. In some aspects, the heterologous moiety comprises albumin, an immunoglobulin constant region or a portion thereof, an immunoglobulin-binding polypeptide, an immunoglobulin G (IgG), albumin-binding polypeptide (ABP), a PASylation moiety, a HESylation moiety, XTEN, a PEGylation moiety, an Fc region, and any combination thereof.

Provided herein is also a nucleic acid sequence encoding the bispecific protein of the present disclosure.

In some aspects, the disclosure includes a vector comprising the nucleic acid molecule.

Provided herein is a host cell comprising the vector of the present disclosure. In some aspects, the host cell is a eukaryotic cell. In some aspects, the host cell is selected from the group consisting of a mammalian cell, an insect cell, a yeast cell, a transgenic mammalian cell, and a plant cell. In some aspects, the host cell is a mammalian cell.

Provided herein is a pharmaceutical composition comprising the bispecific protein of the disclosure, the nucleic acid sequence, the vector, or the host cell and a pharmaceutically acceptable excipient.

Also provided is a kit comprising the bispecific protein, the nucleic acid sequence, the vector, the host cell, or the pharmaceutical composition and instructions for administering the bispecific protein to a subject in need thereof.

The prevent disclosure also provides a method of producing the bispecific protein, comprising culturing the host cell under suitable conditions and recovering the bispecific protein.

Also provided herein is a method of treating a disease or disorder in a subject in need thereof, comprising administering the bispecific protein, the nucleic acid sequence, the vector, the host cell, or the pharmaceutical composition to the subject. In some aspects, the disease or disorder is an immunological disorder. In some aspects, the immunological disorder is an inflammatory disease or an autoimmune disease. In some aspects, the inflammatory disease or autoimmune disease is selected from the group consisting of multiple sclerosis, rheumatoid arthritis, interstitial cystitis, type 1 diabetes, ulcerative colitis, Crohn's disease, psoriasis, psoriatic arthritis, ankylosing spondylitis, hidradenitis suppurativa, prurigo nodularis, non-infectious uveitis, renal/organ transplant, polymyalgia rheumatica, systemic lupus erythematous (SLE), cutaneous lupus erythematous, reduction of cardiovascular risk in CKD patients, gout, depression, inflammatory hand osteoarthritis, hand osteoarthritis, vitiligo, Graves disease, asthma, non-eosinophilic asthma, non-eosinophilic COPD, alopecia areata, COVID-19 related CRS, calcium pyrophosphate deposition disease (psuedogout), CAR-T related CRS, interstitial lung disease (ILD), systemic sclerosis-associated interstitial lung disease (SSc-ILD), connective tissue disease-associated interstitial lung disease (CTD-ILD), Sjogren's syndrome, giant cell arteritis, systemic sclerosis (SSc), dermatomyositis/polymyositis, myasthenia gravis, antiphospholipid syndrome, sarcoidosis, lupus nephritis, IgG4-Related Disease(IgG4-RD), IgA nephroathy, immune thrombocytopenia purpura (ITP), primary sclerosing cholangitis, primary biliary cirrhosis, focal segmental glomerulosclerosis (FSGS), multiple myeloma, pulmonary arterial hypertension, autoimmune hepatitis, Gougerot-sjogren, macrophage activation syndrome, inclusion body myositis, multicentric Castleman's disease, cystoid macular edema, autoimmune myocarditis, pemphigus, bullous pemphigoid, chronic inflammatory demyelinating polyneuropathy, HCV-induced vasculitis, ANCA-associated vasculitis, Guillain Barre syndrome, multifocal motor neuropathy, anti-NMDAR encephalitis, acute graft versus host disease, chronic graft versus host disease, acute disseminated encephalomyelitis (ADEM), acute optic neuritis (AON), neuromyelitis optica (NMO), transverse myelitis, anti-MAG neuropathy, refractory aortitis, autoimmune hemolytic anemia, membranous nephropathy, Behcet's disease, Wegener's granulomatosis, Takayasu's disease, Kawasaki's disease, granulomatosis with polyangiitis, microscopic polyangiitis, Churg-Strauss syndrome, anti-glomerular basement membrane disease, hypocomplementemic urticarial vasculitis, IgA vasculitis (Henoch-Schönlein purpura), polyarteritis nodosa, Lichen planus, Schnitzler's syndrome, anti-MOG neuritis, thyroid eye disease, IL6 driven lymphoproliferative diseases (HLH, Langerhan's), Adult-onset Still's disease (AOSD), juvenile idiopathic arthritis, relapsing polychondritis, chronic urticaria, eosinophilic asthma, food allergy desensitization, celiac disease, allergic rhinitis, allergic conjunctivitis, atopic dermatitis, eosinophilic esophagitis, eosinophilic COPD. In some aspects, the immunological disorder is one or more of immunological deficiency syndromes, which include, but not limited to primary immune deficiency diseases, such as severe combined immunodeficiency (SCID), and acquired immunodeficiency syndrome (AIDS).

Also provided herein is a method of treating an infectious disease in a subject in need thereof, comprising administering the bispecific protein, the nucleic acid sequence, the vector, the host cell, or the pharmaceutical composition to the subject. In some aspects, the infectious disease is caused by one or more of pathogens, which include, but not limited to HIV, hepatitis (A, B, & C), influenza, herpes, giardia, malaria, leishmania, Staphylococcus aureus, Pseudomonas aeruginosa, and COVID-19.

Also provided herein is a method of treating cancer in a subject in need thereof, comprising administering the bispecific protein, the nucleic acid sequence, the vector, the host cell, or the pharmaceutical composition to the subject. In some aspects, the cancer is one or more of bladder cancer, breast cancer, uterine cancer, endometrial carcinoma, ovarian cancer, colorectal cancer, colon cancer, head and neck cancer, lung cancer, stomach cancer, germ cell cancer, bone cancer, squamous cell cancer, skin cancer, neoplasm of the central nervous system, lymphoma, leukemia, sarcoma, virus-related cancer, small-cell lung cancer, non-small cell lung cancer, gastrointestinal cancer, Hodgkin's or non-Hodgkin's lymphoma, pancreatic cancer, glioblastoma, glioma, cervical cancer, ovarian cancer, liver cancer, myeloma, salivary gland carcinoma, kidney cancer, basal cell carcinoma, melanoma, prostate cancer, vulval cancer, thyroid cancer, testicular cancer, esophageal cancer, or head or neck cancer, and any combinations thereof.

Also provided herein is a method of boosting an immune response to an antigen in a subject, comprising administering the bispecific protein, the nucleic acid sequence, the vector, the host cell, or the pharmaceutical composition to the subject. In some aspects, the antigens include, but not limited to tumor antigens or antigens from viruses, bacteria or other pathogens. In some aspects, the protein is administered to the subject sequentially with an additional agent. In some aspects, the protein is administered to the subject concurrently with an additional agent. In some aspects, the additional agent comprises an immunomodulatory agent or a cytokine inhibitor. In some aspects, the cytokine inhibitor targets one or more of IL-1B, IL-la, IL-18, IL-36, IL-37, IL-33, TNFα, LTα, BAFF, APRIL, IL-2, IFN-γ, IL-4, IL-5, IL-17, IL-12, IL-15, IL-21, IL-23, IL-10, IL-22, TGF-β, VEGF, IL-6, IL-10, TGF-β, VEGF, IFN-γ, or any combination thereof. In some aspects, the additional agent comprises an immunosuppressive agent. In some aspects, the immunosuppressive agent comprises glucocorticoids, cyclophosphamide, antimetabolites, methotrexate, azathioprine, mycophenolate mofetil, cyclosporine, tacrolimus, sirolimus, everolimus, or any combination thereof.

In some aspects, the bispecific protein is administered to the subject as a maintenance therapy intended to prevent the occurrence or recurrence of the disease. In some aspects, the bispecific protein is administered via a topical, epidermal mucosal, intranasal, oral, vaginal, rectal, sublingual, topical, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural or intrasternal route.

### BRIEF DESCRIPTION OF THE DRAWINGS

For the purpose of illustrating the disclosure, there are depicted in the drawings certain aspects of the disclosure. However, the disclosure is not limited to the precise arrangements and instrumentalities of the aspects depicted in the drawings.

FIGS. 1A-1B depict a general schematic diagram of certain exemplary bispecific proteins described herein. FIG. 1A illustrates the protein structure of an exemplary bispecific protein; FIG. 1B provides a linear view of the four polypeptides to be expressed to assemble an exemplary bispecific protein.

FIG. 2A shows the results of an exemplary α-IL6R/CTLA-4 bispecific protein (SEQ ID NO: 1 and NO: 3) from an SDS-polyacrylamide gel electrophoresis under non-reducing conditions. The purified, intact bispecific protein detected as Peak 3 has an estimated molecular weight (MW) of 215 kilodaltons. FIG. 2B shows the results of an exemplary α-IL6R/CTLA-4 bispecific protein from an SDS-polyacrylamide electrophoresis under reducing conditions. The disassociated light and heavy chains have apparent MW of 29 (Peak 5) and 91-99 kilodaltons (Peak 7-8), respectively.

FIGS. 3A-3D show the results of an exemplary α-IL6R/CTLA-4 bispecific protein (SEQ ID NO: 1 and NO: 3) from Biacore bind assays using surface plasmon resonance measurements. FIG. 3A provides the sensorgram of an exemplary α-IL6R/CTLA-4 bispecific protein at various concentrations binding to immobilized human CD80. FIG. 3B provides a curve fitting of the CD80 binding at steady state with a bispecific protein at various concentrations. FIG. 3C provides the sensorgram of an exemplary α-IL6R/CTLA-4 bispecific protein binding to immobilized human IL6R from a single-cycle kinetics assay. FIG. 3D provides a summary table showing the parameters of an exemplary α-IL6R/CTLA-4 bispecific protein (Duotein-A) binding to its targets.

FIGS. 4A-4C show the results of an exemplary α-IL6R/CTLA-4 bispecific protein (SEQ ID NO: 1 and NO: 3) from a mixed leukocyte reaction assay. FIG. 4A and FIG. 4B show the T cell proliferation induced by allo-reaction in the presence of indicated agents. Duotein is an exemplary α-IL6R/CTLA-4 bispecific protein, and abatacept, an CTLA4-Ig serve as a control.

FIG. 4C shows a dose-dependent inhibition of IL2 secretion in a mixed leukocyte co-culture by an exemplary α-IL6R/CTLA-4 bispecific protein.

FIGS. 5A-5B show the results of an exemplary α-IL6R/CTLA-4 bispecific protein (Duotein) from a murine model of allogeneic bone marrow transplant induced-GvHD. FIG. 5A shows administration of the α-IL6R/CTLA-4 bispecific protein that binds to murine IL6R and CD80 significantly reduced clinical severity of GvHD compared to vehicle control, anti-IL6R antibody, or CTLA4-Ig treated animals. FIG. 5B shows that mice treated with α-IL6R/CTLA-4 bispecific protein have higher chance of survival after induced-GvHD.

FIGS. 6A-6B show the results of an exemplary α-IL6R/CTLA-4 bispecific protein (SEQ ID NO: 1 and NO: 3) from a pharmacokinetics study in cynomolgus monkeys. The concentration of α-IL6R/CTLA-4 bispecific protein in the serum from peripheral blood was measured at indicated time points after i.v. injection at a dose of 1 mg/kg and 10 mg/kg, respectively (n=3 each).

FIGS. 7A-7B shows the QC results of an exemplary α-CD40/CTLA-4 bispecific protein (SEQ ID NO: 2 and NO: 11) by HPLC-SEC and SDS-PAGE. The purity of bispecific protein indicated as the main peak in FIG. 7A is above 99%. FIG. 7B shows the bispecific protein has an estimated molecular weight of about 200 kilodaltons by SDS-PAGE under non-reducing conditions. Under reducing conditions, the light and heavy chains have apparent MW of 25 and 75 kilodaltons, respectively.

FIGS. 8A-8C show the results of an exemplary α-CD40/CTLA-4 bispecific protein (SEQ ID NO: 2 and NO: 11) from Biacore bind assays using surface plasmon resonance measurements. FIG. 8A provides the sensorgram of an exemplary α-CD40/CTLA-4 bispecific protein at various concentrations binding to immobilized human CD80. FIG. 8B provides a curve fitting of the CD80 binding at steady state with an exemplary α-CD40/CTLA-4 bispecific protein at various concentrations. FIG. 8C provides a summary table showing the parameters of an exemplary α-CD40/CTLA-4 bispecific protein (Duotein-B) binding to its targets.

FIGS. 9A-9C show the results of an exemplary α-CD40/CTLA-4 bispecific protein (SEQ ID NO: 2 and NO: 11) from mixed leukocyte reaction assays. FIG. 9A shows the T cell proliferation induced by allo-reaction in the presence of indicated agents. Duotein-B is an exemplary α-CD40/CTLA-4 bispecific protein; Duotein-αCD40 is a control bispecific protein with the same CD40-binding motif but a suboptimal binding to CD80; Duotein-CTLA4 is a control bispecific protein with the same CD80 binding motif but without the CD40-binding capability; Abatacept is a CTLA4-Ig serving as a positive control. FIG. 9B shows Duotein-B achieved a more profound inhibition of T cell proliferation than a combination of two control bispecific proteins for single targets at an equal concentration of 100 μg/mL. FIG. 9C shows the IL2 secretion in a mixed leukocyte co-culture in the presence of indicated bispecific proteins at concentrations of 100 μg/mL.

### DETAILED DESCRIPTION

Described herein are novel dual-targeting immunomodulatory antibodies and bispecific proteins to address the shortcomings of existing treatments for immunological disorders. In some aspects, the disclosure provides a bispecific protein that binds to two different targets that are each involved in the pathogenesis of an immunological disorder through a distinct pathway, whereby biological effects of the two targets can be modulate at the same time to achieve a synergistic therapeutic effect. Compared to existing treatment options, the dual-targeting approach described herein has the following advantages. First, by simultaneously addressing multiple pathogenic factors, it is thereby likely to result in a clinical response in a broader basis of patients. Second, by a careful selection of the two targets, it can address two complementary pathways, innate immunity and adaptive immunity for example, at the same time to achieve better efficacy while maintaining a desired safety profile. Third, the bispecific protein can exhibit markedly increased affinity (or avidity) and target residence time when both binding motifs bind simultaneously to their target sites. This is because binding of one binding motif forces the second one to stay close to its corresponding site, since the two binding motifs are physically linked. This ‘forced proximity’ favors its binding and rebinding (once dissociated) to that site. For carefully selected targets, the dual-targeting approach will improve target engagement with higher affinity and slower dissociation than the mono-specific drugs for each target that are dosed separately. This can potentially translate to a high target selectivity and long duration of action. All these features can lead to a more effective therapy without increasing the treatment burden, doubling the cost, and any delay for patients to receive optimal treatment.

In other aspects, the disclosure provides a bispecific protein composed of one motif with immunomodulatory property connected to a second motif that binds to a protein expressed on the surface of certain cell types. This approach describe herein can deliver an immunomodulator specifically to certain cell types to achieve higher potency within those cells and to avoid undesired effects on the bystander cells. All these features can lead to a more effective therapy with increased specificity for desired biological effects and an improved safety profile.

Before the present disclosure is described in greater detail, it is to be understood that this disclosure is not limited to the particular compositions or process steps described, as such can, of course, vary. As will be apparent to those of skill in the art upon reading this disclosure, each of the individual aspects described and illustrated herein has discrete components and features which can be readily separated from or combined with the features of any of the other several aspects without departing from the scope or spirit of the present disclosure. Any recited method can be carried out in the order of events recited or in any other order that is logically possible.

The headings provided herein are not limitations of the various aspects of the disclosure, which can be defined by reference to the specification as a whole. It is also to be understood that the terminology used herein is for the purpose of describing particular aspects only, and is not intended to be limiting.

### I. Definitions

In order that the present disclosure can be more readily understood, certain terms are first defined. As used in this application, except as otherwise expressly provided herein, each of the following terms shall have the meaning set forth below. Additional definitions are set forth throughout the application.

Throughout this disclosure, the term “a” or “an” entity refers to one or more of that entity; for example, “a chimeric polypeptide,” is understood to represent one or more chimeric polypeptides. As such, the terms “a” (or “an”), “one or more,” and “at least one” can be used interchangeably herein. In addition, “or” is used mean an open list of the components in the list. For example, “wherein X comprises A or B” means X comprises A, X comprises B, X comprises A and B, or X comprises A or B and any other components.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range, unless otherwise explicitly stated.

Abbreviations used herein are defined throughout the present disclosure. Various aspects of the disclosure are described in further detail in the following subsections.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10% (e.g., a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value)). For example, “about 10,” as used herein, includes 9 to 11. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As used herein, the term “approximately,” as applied to one or more values of interest, refers to a value that is similar to a stated reference value. In some aspects, the term “approximately,” like the term “about,” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As described herein, any concentration range, percentage range, ratio range or integer range is to be understood to include the value of any integer within the recited range and, when appropriate, fractions thereof (such as one tenth and one hundredth of an integer), unless otherwise indicated.

Amino acids may be referred to herein by either their commonly known three letter symbols or by the one-letter symbols recommended by the IUP AC-IUB Biochemical Nomenclature Commission. Nucleotides, likewise, may be referred to by their commonly accepted single-letter codes.

The numbering of amino acids in the variable domain, complementarity determining region (CDRs) and framework regions (FR), of an antibody follow, unless otherwise indicated, the Kabat definition as set forth in Kabat et al. Sequences of Proteins of Immunological Interest, 5th Ed. Public Health Service, National Institutes of Health, Bethesda, MD. (1991). Using this numbering system, the actual linear amino acid sequence may contain fewer or additional amino acids corresponding to a shortening of, or insertion into, a FR or CDR of the variable domain. For example, a heavy chain variable domain may include a single amino acid insertion (residue 52a according to Kabat) after residue 52 of H2 and inserted residues (e.g. residues 82a, 82b, and 82c, etc according to Kabat) after heavy chain FR residue 82. The Kabat numbering of residues may be determined for a given antibody by alignment at regions of homology of the sequence of the antibody with a “standard” Kabat numbered sequence. Maximal alignment of framework residues frequently requires the insertion of “spacer” residues in the numbering system, to be used for the Fv region. In addition, the identity of certain individual residues at any given Kabat site number may vary from antibody chain to antibody chain due to interspecies or allelic divergence.

As used herein, the terms “antibody” and “antibodies”, also known as immunoglobulins, encompass monoclonal antibodies (including full-length monoclonal antibodies), polyclonal antibodies, multispecific antibodies formed from at least two different epitope binding fragments (e.g., multispecific antibodies), human antibodies, humanized antibodies, camelised antibodies, single-chain Fvs (scFv), single-chain antibodies, single domain antibodies, domain antibodies, Fab fragments, F(ab′)2 fragments, antibody fragments that exhibit the desired biological activity (e.g. the antigen binding portion), disulfide-linked Fvs (dsFv), and anti-idiotypic (anti-Id) antibodies (including, e.g., anti-Id antibodies to antibodies of the invention), intrabodies, and epitope-binding fragments of any of the above. In particular, antibodies include immunoglobulin molecules and immunologically active fragments of immunoglobulin molecules, i.e., molecules that contain at least one antigen-binding site. Antibodies also include peptide fusions with antibodies or portions thereof such as a protein fused to an Fc domain. Immunoglobulin molecules can be of any isotype (e.g., IgG, IgE, IgM, IgD, IgA and IgY), subisotype (e.g., IgG1, IgG2, IgG3, IgG4, IgA1 and IgA2) or allotype (e.g., Gm, G1m(f, z, a or x), G2m(n), G3m(g, b, or c), Am, Em, and Km(1, 2 or 3)). Antibodies may be derived from any mammal, including, but not limited to, humans, monkeys, pigs, horses, rabbits, dogs, cats, mice, etc., or other animals such as birds (e.g. chickens).

As used herein, an “antigen” refers to a molecule to which an antibody (or antigen binding fragment thereof) can selectively bind. The target antigen may be a protein, carbohydrate, nucleic acid, lipid, hapten, or other naturally occurring or synthetic compound. An antigen may also be administered to an animal to generate an immune response in the animal.

By “binding affinity” is meant the strength of the total noncovalent interactions between a single binding site of a molecule (e.g., an antigen binding fragment or a receptor) and its binding partner (e.g., an antigen/antigenic peptide or a ligand). The affinity of a molecule X for its partner Y can generally be represented by the dissociation constant (K_{D}). Affinity can be measured by standard methods known in the art, including those described herein. A low-affinity complex contains a molecule that generally tends to dissociate readily from its partner, whereas a high-affinity complex contains a molecule that generally tends to remain bound to its partner for a longer duration.

The term “binding domain” or “BD” refers to a polypeptide (e.g., an antibody, an antigen binding fragment, and/or a ligand binding domain of a cellular receptor) that binds to an epitope or region within a target polypeptide, e.g., a receptor.

The term “immune response” is as understood in the art, and generally refers to a biological response within a vertebrate against foreign agents or abnormal, e.g., cancerous cells, which response protects the organism against these agents and diseases caused by them. An immune response is mediated by the action of one or more cells of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell, a Th cell, a CD4⁺ cell, a CD8⁺ T cell, or a Treg cell, or activation or inhibition of any other cell of the immune system, e.g., NK cell.

The term “immunomodulator” or “immunoregulator” refers to an agent, e.g., an agent targeting a component of a signaling pathway that can be involved in modulating, regulating, or modifying an immune response. “Modulating,” “regulating,” or “modifying” an immune response refers to any alteration in a cell of the immune system or in the activity of such cell (e.g., an effector T cell, such as a Th1 cell, Th2 cell, or CD8+ T cell). More particularly, as used herein, the term “modulating” includes inducing, inhibiting, potentiating, elevating, increasing, or decreasing a given activity or response. Such modulation includes stimulation or suppression of the immune system which can be manifested by an increase or decrease in the number of various cell types, an increase or decrease in the activity of these cells, or any other changes which can occur within the immune system. Both inhibitory and stimulatory immunomodulators have been identified and implicated in the pathogenesis of various immunological disorders. In some embodiments, the immunomodulator targets a molecule on the surface of a T cell. An “immunomodulatory target” or “immunoregulatory target” is a molecule, e.g., a cell surface molecule, that is targeted for binding by, and whose activity is altered by the binding of, a substance, agent, moiety, compound or molecule. Immunomodulatory targets include, for example, receptors on the surface of a cell (“immunomodulatory receptors”) and receptor ligands (“immunomodulatory ligands”).

As used herein, the term “effector cell” means an immune cell which is involved in the effector phase of an immune response, as opposed to the cognitive and activation phases of an immune response. Exemplary immune cells include a cell of a myeloid or lymphoid origin, e.g., lymphocytes (e.g., B cells, T cells including helper T (Th) cells and cytolytic T cells (CTLs), and natural killer cells), myeloid cells (e.g., dendritic cells, macrophages, monocytes, eosinophils, neutrophils, basophils and mast cells). Effector cells express specific Fc receptors and carry out specific immune functions. An effector cell can induce antibody-dependent cell-mediated cytotoxicity (ADCC) or antibody-dependent cell mediated phagocytosis (ADCP). For example, natural killer cells, macrophages, dendritic cells, neutrophils, and eosinophils which express FcγR are involved in specific killing of target cells and presenting antigens to other components of the immune system.

As used herein, the term “Treg” or “Regulatory T cells” means a specialized subpopulation of T cells that act to suppress immune response, thereby maintaining homeostasis and self-tolerance. It has been shown that Tregs are able to inhibit T cell proliferation and cytokine production and play a critical role in preventing autoimmunity. Different subsets with various functions of Treg cells exist. Tregs can be usually identified by flow cytometry. The most specific marker for these cells is FoxP3, which is localized intracellularly. Selected surface markers such as CD25^{high} (high molecular density) and CD127^{low} (low molecular density) could serve as surrogate markers to detect Tregs in a routine clinical practice. Dysregulation in Treg cell frequency or functions may lead to the development of autoimmune disease. Therapeutical Treg modulation is considered to be a promising therapeutical approach to treat some selected disorders, such as allergies, and to prevent allograft rejection.

As used herein, the term “pharmaceutically-acceptable carrier” is intended to include any and all solvents, dispersion media, coatings, antibacterial and antifungal compounds, isotonic and absorption delaying compounds, and the like, compatible with pharmaceutical administration. Pharmaceutically-acceptable carriers and their formulations are known to one skilled in the art and are described, for example, in Remington's Pharmaceutical Sciences (20th edition, ed. A. Gennaro, 2000, Lippincott, Williams & Wilkins, Philadelphia, PA.).

As used herein, the term “polynucleotide” or “nucleic acid” means any RNA or DNA, which may be unmodified or modified RNA or DNA. Polynucleotides include, without limitation, single- and double-stranded DNA, DNA that is a mixture of single- and double stranded regions, single- and double-stranded RNA, RNA that is mixture of single- and double-stranded regions, and hybrid molecules comprising DNA and RNA that may be single-stranded or, more typically, double-stranded or a mixture of single- and double stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified for stability or for other reasons.

As used herein, the terms “polypeptide,” “peptide” and “protein” are used interchangeably herein to mean a polymer comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art. Such modifications are well described in basic texts and in more detailed monographs, as well as in a voluminous research literature.

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), rectally, intrathecally, intratumorally or topically. Administration includes self-administration and the administration by another.

As used herein, the terms “individual”, “patient”, or “subject” can be an individual organism, a vertebrate, a mammal, or a human. In some embodiments, the individual, patient or subject is a human.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

Amino acid sequence modification(s) of the bispecific proteins described herein are contemplated. For example, it may be desirable to increase or decrease the binding affinity and/or other biological properties of the protein. Amino acid sequence variants of a protein are prepared by introducing appropriate nucleotide changes into its corresponding nucleic acid, or by peptide synthesis. Such modifications include, for example, deletions from, and/or insertions into and/or substitutions of, residues within the amino acid sequences of the bispecific protein. Any combination of deletion, insertion, and substitution is made to obtain the bispecific protein of interest, as long as the obtained protein possesses the desired properties. The modification also includes the change of the pattern of glycosylation of the protein. The sites of greatest interest for substitutional mutagenesis include the hypervariable regions of the antigen binding fragments of the bispecific proteins of the present technology are also contemplated. “Conservative substitutions” are shown in the Table 1 below.

TABLE 1
Conservative Amino Acid Substitutions

| Amino Acid | Conservative Substitutions |
|---|---|
| G | A, S, N |
| P | E |
| D | S, K, Q, H, N, E |
| E | P, D, S, R, K, Q, H. N |
| N | G, D, E, T, S, R, K, Q, H |
| H | D, E, N, M, R, Q |
| Q | D, E, N, H, M, S, R, K |
| K | D, E, N, Q, R |
| R | E, N, H, Q, K |
| S | G, D, E, N, Q, A, T |
| T | N, S, V, A |
| A | G, S, T, V |
| M | H, Q, Y, F, L, I, V |
| V | T, A, M, F, L, I |
| I | M, V, Y, F, L |
| L | M, V, I, Y, F |
| F | M, V, I, L, W, Y |
| Y | H, M, I, L, F, W |
| W | F, Y |
| C | None |

### II. Dual-Targeting Immunomodulatory Antibodies and Fusion Proteins (Bispecific Proteins)

### IIA. Design and Configuration

The disclosure provides bispecific proteins that bind two targets (e.g., IL6R and CD80/CD86 or CD40 and CD80/CD86) and that are bivalent for binding to each of the first and second targets. Exemplary configurations of these novel binding proteins disclosed herein are also referred to as “Duotein” or “Duoteins”.

In some aspects, the disclosure provides a bispecific protein comprising: a first binding domain (BD1, e.g., anti-IL6R antibody or anti-CD40 antibody) that binds to a first target, a second binding domain (BD2, e.g., CTLA-4 ectodomain) that binds to a second target, and an Fc region having CH2 and CH3 domain; wherein the Fc region includes BD2 at a solvent exposed loop in the CH2 domain, the CH3 domain, or at the interface of the CH2 and CH3 domains, or being connected to the C-terminal of CH3 domain; and wherein the protein is bivalent for binding to each of the first and second targets.

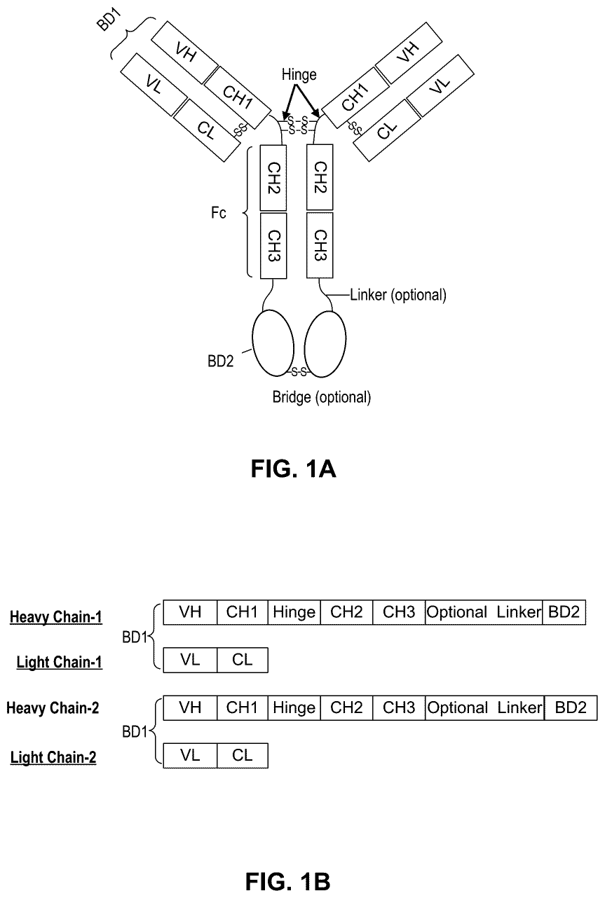

In some aspects, the disclosure provides bispecific proteins having a structure comprising domains that are generally illustrated by the schematic diagrams in FIG. 1A, where BD1 (e.g., anti-IL6R antibody or anti-CD40 antibody) is connected to the Fc region via an antibody hinge region; BD2 (e.g., CTLA-4 ectodomain) is connected to the C-terminal of the CH3 domain of Fc region through a polypeptide linker. In some aspects, BD2 (e.g., CTLA-4 ectodomain) is connected to the Fc region at a site in the CH2 domain, the CH3 domain, or at the interface of the CH2 and CH3 domains.

In some aspects, the disclosure provides a general structure of bispecific proteins that comprises two chimeric heavy chains, each comprising a heavy chain variable region (VH), a heavy chain constant region 1 (CH1), a hinge or poly peptide linker region, an Fc region comprising a CH2 domain and a CH3 domain, wherein a second binding domain (BD2, e.g., CTLA-4 ectodomain) is connected to the C-terminal of the CH3 domain of Fc region directly or through a polypeptide linker (L1 and/or L2). The structure of this aspect of the disclosure also comprises two conventional antibody light chains, each comprising a light chain variable region (VL) and light chain constant region (CL), which forms part of the first binding domain (BD1, e.g., anti-IL6R antibody or anti-CD40 antibody). The linear configuration of each heavy chain and light chain in these specific embodiments is illustrated by the schematic diagrams in FIG. 1B.

In some aspects, a bispecific protein disclosed herein can comprise two heavy-light chain pairs derived from a specific binding protein (e.g., anti-IL6R antibody or anti-CD40 antibody), wherein the heavy and light chains each comprise a variable region (e.g., VL and VH), which together form a first binding unit, and wherein the heavy chains each further comprises a second binding unit. Where the first and second binding units bind different epitopes, each heavy-light chain pair is bispecific and the two pairs together are bivalent for each epitope. Where the first and second binding units bind the same epitope, each heavy-light chain pair is monospecific and the two pairs together are tetravalent for the epitope. In some aspects, the two heavy-light chain pairs are identical. In some aspects, the two heavy-light chain pairs are not identical.

In some aspects, the domains of a bispecific protein disclosed herein can be based on known immunoglobulin domains. Immunoglobulin molecules such as monoclonal antibodies (mAbs) are widely used as diagnostic and therapeutic agents, and methods for engineering the binding fragments of mAbs are well-known in the art. Monoclonal antibodies, like all immunoglobulin molecules, are made up of heavy chain and light chain peptide subunits, which each include variable and constant domains that confer binding specificity (variable domain) and isotype (constant domain).

In some aspects, the bispecific protein disclosed herein can have a similar overall structure to a conventional antibody, but are distinguishable by the presence of an additional binding unit that is attached at a location within the Fab domain, attached at a location away from the Fab domain and within the Hinge or Fc regions (e.g., within the CH2, CH3, or CH4 regions, or at the interface of such regions such as the CH2-CH3 interface). Thus, unlike conventional antibodies that are bivalent for binding to a single epitope, the bispecific protein is bivalent for binding to two epitopes. However, as described herein, the bispecific protein can still maintain numerous desirable properties of conventional antibodies, such as ability to bind FcRn.

### IIB. Binding Domains

In some aspects, the disclosure provides a bispecific protein that binds to two different targets (e.g., IL6R and CD80/CD86 or CD40 and CD80/CD86) that are each involved in the pathogenesis of an immunological disorder through a distinct pathway, whereby biological effects of the two targets can be modulate at the same time to achieve a synergistic therapeutic effect when the bispecific protein is administered to a subject (e.g., a human subject). For example, many immunological disorders involve both innate and adaptive immunity, with each amplifying the signals that activate the other. By targeting both innate and adaptive immunity at the same time with the bispecific protein, the treatment can inhibit the positive feedback loop and thereby prevent pathogenic immune responses, leading to a greater therapeutic effect than addressing each target alone.

In some aspects, the disclosure provides a bispecific protein that binds to two different targets (e.g., IL6R and CD80/CD86 or CD40 and CD80/CD86) that are each involved in the pathogenesis of an immunological disorder through the same pathway, whereby biological effects of the two targets can be modulated at the same time to achieve a greater therapeutic effect than modulating any of the two targets alone when the bispecific protein is administered to a subject. For example, many immunological disorders involve a signaling cascade which eventually lead to a catastrophic immune response that causes the disease. It is often difficult to sufficiently block the signaling by targeting one component on the cascade due to a leakage. By targeting two components on the cascade at the same time with the bispecific protein, the treatment can effectively block the signaling and achieve a greater therapeutic effect than addressing each target alone.

In some aspect, the disclosure provides a bispecific protein that binds to two different targets (e.g., IL6R and CD80/CD86 or CD40 and CD80/CD86) and exhibit markedly increased affinity and target residence time through a mechanism of forced proximity when both binding motifs bind simultaneously to their target sites. This feature can potentially translate to a high target selectivity and long duration of action, which lead to an unexpected, greater therapeutic effect than addressing each target alone or addressing both with a combination of two separate monospecific therapeutics.

In some aspects, the first binding domain (BD1) is an antigen binding portion that binds to IL6R or CD40, for example, a Fab fragment of a conventional monoclonal antibody or a recombinantly produced antigen binding fragment comprising a variable light chain (VL), a constant light chain (CL), a variable heavy chain (VH), and a constant heavy chain portion (CH1). Optionally, the light and heavy chains of the Fab may be interconnected via one or more disulfide linkages such as, for example, via a suitable antibody hinge region.

In some aspects, the Fab that binds to IL6R or CD40 is derived from or based on the sequence of a conventional monoclonal antibody, such as a conventional murine, humanized, or human antibody. In some aspects, the bispecific protein containing the Fab derived from or based on the sequence of a conventional monoclonal antibody (e.g., anti-IL6R antibody or anti-CD40 antibody) retains one or more functional activities of the conventional antibody (e.g., retains at least 80% or more (80%, 85%, 90%, 95%, 97%, 98%, 99% or 100%) of a functional activity). For example, in some aspects, the bispecific protein containing such a Fab retains the affinity for one or more antigens, inhibitory activity, modulatory activity on immune system, ability to activate or induce an immune response.

In some aspects, the bispecific protein disclosed comprises a second binding domain (BD2) that binds CD80/CD86. The binding unit 2 (or binding domain 2 (BD2)) may be associated with the BD1 via an amino acid linker, or covalent bonding through recombinant expression of BD2 within the Fab region, within the hinge region, or within the Fc region at the CH2, CH3, or interface of CH2 and CH3, or at the C-terminal of the CH3 domain.

In some aspects, BD2 that binds to CD80/CD86 is an scFv, for example, an scFv derived from a conventional monoclonal antibody comprising a variable light chain (VL) and a variable heavy chain (VH) interconnected by a flexible linker, such as a glycine-serine linker. Optionally, the variable light and variable heavy chains of the scFv may be further interconnected via one or more disulfide linkages, and as described above, may include one or more mutations or variations. In some aspects, the scFv is derived from or based on the sequence of a conventional monoclonal antibody, such as a conventional murine, humanized or human antibody. In some aspects, the scFv is derived from or based on the sequence of a conventional monoclonal antibody retains one or more functional activities of the conventional antibody (e.g., retains at least 80% or more (80%, 85%, 90%, 95%, 97%, 98%, 99% or 100%) of a functional activity). For example, in some aspects, the scFv retains the affinity for one or more antigens, inhibitory activity, modulatory activity on immune system, ability to activate or induce an immune response.

In some aspects, the disclosure provides a bispecific protein comprising: BD1 and BD2 that bind to IL6R and CD80/CD86, respectively. In some aspects, the bispecific protein that binds to IL6R and CD80/CD86 has antagonistic effects on the signaling of IL6 and T cell activation.

In some aspects, the disclosure provides a bispecific protein comprising: BD1 and BD2 that bind to CD40 and CD80/CD86, respectively. In some aspects, the bispecific protein that binds to CD40 and CD80/CD86 has antagonistic effects on the signaling of CD40 and T cell activation

In some aspects, the bispecific protein that binds to IL6R or CD40 and CD80/CD86 comprises (i) a first polypeptide chain comprising, from N-terminus to C-terminus VH-CH1-CH2-CH3-L1-BD2; VH-CH1-CH2-CH3(N-term)-L1-BD2-L2-CH3(C-term); VH-CH1-CH2-L1-BD2-L2-CH3; or VH-CH1-CH2(N-term)-L1-BD2-L2-CH2(C-term)-CH3; and (ii) a second polypeptide chain comprising VL-CL1, wherein the VH and VL binds to IL6R or CD40, wherein L1 and L2 are optional linkers.

In some aspects, the bispecific protein comprises two polypeptide chains, three polypeptide chains or four polypeptide chains. In some aspects, the bispecific protein comprises two identical first polypeptide chains and two identical second polypeptide chains. In some aspects, the bispecific protein comprises two first polypeptide chains (PPT-1 and PPT-2) that are different and/or two second polypeptide chains that are different. In some aspects, the PPT-1 comprises a CH2-CH3 domain comprising T366W/S354C substitutions and the PPT-2 comprises a CH2-CH3 domain comprising T366S/L368A/Y407V/Y349C substitutions.

In some aspects, the bispecific protein further comprises a heterologous moiety fused to the BD1 and/or the BD2. In some aspects, the heterologous moiety is a half-life extending moiety. In some aspects, the heterologous moiety comprises a non-polypeptide moiety. In other aspects, the heterologous moiety comprises a polypeptide. In some aspects, the heterologous moiety comprises albumin, an immunoglobulin constant region or a portion thereof, an immunoglobulin-binding polypeptide, an immunoglobulin G (IgG), albumin-binding polypeptide (ABP), a PASylation moiety, a HESylation moiety, XTEN, a PEGylation moiety, an Fc region, and any combination thereof.

Several methodologies can be used alone or in combination to improve the stability of a bispecific protein disclosed herein. One potential methodology that can be used, alone or in combination with one or more of the other methodologies described herein, is engineered the length and/or composition of the linker connecting BD2 (e.g., CTLA-4 ectodomain). Another methodology that can be used is to introduce at least two amino acid substitutions (also referred to as modifications or mutations), typically to introduce a cysteine, into the C-terminal of BD2 (e.g., CTLA-4 ectodomain) so as to promote disulfide bond formation. Additional methodology that can be used is to vary the length of BD2 (e.g., CTLA-4 ectodomain) or to select certain truncated form to improve the protein stability. A further method can be used is to introduce one or more stabilizing mutations by mutating one or more surface residues of BD2 (e.g., CTLA-4 ectodomain). All these methods can be used alone or in combination with one or more of the other methodologies described herein.

Another method that can be used, alone or in combination with one or more of the other methods described herein, is to introduce one or more amino acid substitutions by mutating one or more residues present in BD1 (e.g., anti-IL6R antibody or anti-CD40 antibody) and BD2 (e.g., CTLA-4 ectodomain) to increase or decrease their affinity to the target epitopes. The substitutions can include conservative and non-conservative ones.

Yet another method that can be used, on its own or in combination with other methods, is to introduce one or more amino acid substitutions by mutating one or more residues present in BD1 (e.g., anti-IL6R antibody or anti-CD40 antibody) and BD2 (e.g., CTLA-4 ectodomain) to reduce immunogenicity in certain species, including humans.

### IIB(i). CD80 CD86 Binding Domain

In some aspects, the CD80/CD86 binding domain (BD2) comprises a scFv. In some aspects, the BD2 comprises one or more ligand binding domain of a cellular receptor.

In some aspects, the BD2 comprises an ectodomain of CTLA4.

Cytotoxic T-lymphocyte-associated protein 4 (CTLA-4) is expressed on activated T cells and serves as a co-inhibitor to keep T-cell responses in check following CD28-mediated T cell activation. CTLA-4 is believed to regulate the amplitude of the early activation of naive and memory T cells following TCR engagement and to be part of a central inhibitory pathway that affects both antitumor immunity and autoimmunity. CTLA-4 is expressed exclusively on T cells, and the expression of its ligands CD80 (B7.1) and CD86 (B7.2), is largely restricted to antigen-presenting cells, T cells, and other immune mediating cells.

The human CTLA-4 sequence is known as Cytotoxic T-lymphocyte protein 4 (CTLA-4) or CD152 and is shown below:

(SEQ ID NO: 67)
MACLGFQRHKAQLNLATRTWPCTLLFFLLFIPVFCKAMHVAQPAVVLASS
RGIASFVCEYASPGKATEVRVTVLRQADSQVTEVCAATYMMGNELTFLDD
SICTGTSSGNQVNLTIQGLRAMDTGLYICKVELMYPPPYYLGIGNGTQIY
VIDPEPCPDSDFLLWILAAVSSGLFFYSFLLTAVSLSKMLKKRSPLTTGV
YVKMPPTEPECEKQFQPYFIPIN

The exemplary sequences of CTLA-4 useful for the bispecific protein are shown in SEQ ID NOs: 35-40. In some aspects, the ectodomain of CTLA-4 comprises an amino acid sequence having at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to SEQ ID NOs: 35-40, wherein the amino acid sequence is capable of binding to CD80/CD86. In some aspects, wherein the ectodomain of CTLA-4 comprises an amino acid sequence comprising SEQ ID NO: 41 (MYPPPY), wherein the amino acid sequence is capable of binding to CD80 and CD86.

### IIB(ii) IL6R Binding Domain

Interleukin-6 (IL6) is an important cytokine both for innate and adaptive immunity. Numerous cell types produce IL6, including monocytes, T cells, fibroblasts and endothelial cells, especially at inflammation sites. IL6 exerts its biological actions through two main pathways, termed classic signaling and trans-signaling. IL6 classic signaling involves binding of IL6 to the membrane-bound IL6 receptor (mIL6R), which is expressed by a restricted group of cells, including some immune cells, airway epithelial cells and hepatocytes. IL6 binding to mIL6R triggers the dimerization of the signal transducer gp130, which activates the intracellular signal pathways leading the expression of IL6 responsive genes. IL6 trans-signaling is similar in that the same intracellular pathways are activated, but through a different mechanism, where IL6 binds to a soluble version of IL6R (sIL6R), and then the complex is recognized by gp130, leading to IL6-dependent activation of cells that do not express mIL6R. The IL6 pathway is involved in various inflammatory diseases and could be a potential target in a broad spectrum of indications.

In some aspects, the bispecific protein comprises an IL6R binding molecule. In some aspects, the IL6R binding molecule comprises an antibody that specifically binds to IL6R or antigen binding portion thereof. In some aspects, the anti-IL6R antibody or antigen binding portion thereof specifically binds to the same epitope as tocilizumab. In some aspects, the anti-IL6R antibody or antigen binding portion thereof cross competes with tocilizumab.

In some aspects, the IL6R binding molecule (BD1) comprises a heavy chain immunoglobulin variable domain (VH) and a light chain immunoglobulin variable domain (VL) that bind to IL6R, wherein:
(a) the VH comprises a VH-CDR1 sequence of SDHAWS (SEQ ID NO: 19), a VH-CDR2 sequence of YISYSGITTYNPSLKS (SEQ ID NO: 20), and a VH-CDR3 sequence of SLARTTAMDY (SEQ ID NO: 21), and
(b) the VL comprises a VL-CDR1 sequence of RASQDISSYLN (SEQ ID NO: 22), a VL-CDR2 sequence of YTSRLHS (SEQ ID NO: 23), and a VL-CDR3 sequence of QQGNTLPYT (SEQ ID NO: 24).

In some aspects, the IL6R binding domain comprises a VH and a VL, wherein the VH comprises an amino acid sequence that has at least about 80%, at least about 85%, at least about 95%, at least about 96%, at least about 97%, at least about 98%, at least about 99%, or about 100% sequence identity to SEQ ID NO: 25; and/or the VL comprises an amino acid sequence that has at least about 80%, at least about 85%, at least about 95%, at least about 96%, at least about 97%, at least about 98%, at least about 99%, or about 100% sequence identity to SEQ ID NO: 26.

In some aspects, the IL6R binding domain comprises a heavy chain and a light chain, wherein the heavy chain (HC) is selected from the group consisting of the amino acid sequence as set forth in SEQ ID NOs: 3 to 10, or a variant thereof having one or more conservative amino acid substitutions, and a light chain (LC) comprises the amino acid sequence as set forth in SEQ ID NO: 1, or a variant thereof having one or more conservative amino acid substitutions. In some aspects, the heavy chain comprises an amino acid sequence having at least about 95% sequence identity to the HC sequence as set forth in any one of SEQ ID NOs: 3 to 10; and/or the light chain comprises an amino acid sequence having at least 95% sequence identity to the LC sequence as set forth in SEQ ID NO: 1.

Tocilizumab is described in U.S. Pat. No. 5,670,373, published Sep. 23, 1997, and 5,795,965, published Aug. 18, 1998, which are incorporated herein by reference in their entireties.

The anti-IL6R antibodies useful for the bispecific protein are not limited to tocilizumab. Other anti-IL6R antibodies that can be used to construct the bispecific protein include sarilumab, satralizumab, olokizumab, and vobarilizumab. In some aspects, sarilumab can be found in U.S. Pat. No. 7,582,298, published in Dec. 6, 2007, which is incorporated herein by reference in its entirety; satralizumab can be found in U.S. Pat. No. 8,562,991, published in Apr. 28, 2011, which is incorporated herein by reference in its entirety; olokizumab is described in Shaw, Stevan et al. “Discovery and characterization of olokizumab: a humanized antibody targeting interleukin-6 and neutralizing gp130-signaling.” mAbs vol. 6,3 (2014): 774-82. doi:10.4161/mabs.28612, which is incorporated herein by reference in its entirety. Vobarilizumab can be found in PCT/EP2017/070045, published as WO2018029182A1 on Feb. 15, 2018, which is incorporated herein by reference in its entirety. In some aspects, the heavy chain and/or the light chain in the bispecific protein comprises an amino acid sequence having at least about 80%, at least about 85%, at least about 95%, at least about 96%, at least about 97%, at least about 98%, at least about 99%, or about 100% sequence identity to the sequence as set forth in any one of SEQ ID NOs: 42 to 50. Exemplary sequences of VH and VL of anti-IL6R antibodies useful for the bispecific protein are shown in SEQ ID NOs: 42-50.

In some aspects, the bispecific protein comprises a heavy chain immunoglobulin variable domain (VH) comprising an amino acid sequence having at least about 80%, at least about 85%, at least about 95%, at least about 96%, at least about 97%, at least about 98%, at least about 99%, or about 100% sequence identity to SEQ ID NO: 42. In some aspects, the sequence of the variable heavy chain of tocilizumab is shown in SEQ ID NO: 42.

In some aspects, the bispecific protein comprises a light chain immunoglobulin variable domain (VL) comprising an amino acid sequence having at least about 80%, at least about 85%, at least about 95%, at least about 96%, at least about 97%, at least about 98%, at least about 99%, or about 100% sequence identity to SEQ ID NO: 43. In some aspects, the sequence of the tocilizumab variable light chain is shown in SEQ ID NO: 43.

In some aspects, the bispecific protein comprises a heavy chain immunoglobulin variable domain (VH) comprising an amino acid sequence having at least about 80%, at least about 85%, at least about 95%, at least about 96%, at least about 97%, at least about 98%, at least about 99%, or about 100% sequence identity to SEQ ID NO: 44. In some aspects, the sequence of the variable heavy chain of sarilumab is shown in SEQ ID NO: 44.

In some aspects, the bispecific protein comprises a light chain immunoglobulin variable domain (VL) comprising an amino acid sequence having at least about 80%, at least about 85%, at least about 95%, at least about 96%, at least about 97%, at least about 98%, at least about 99%, or about 100% sequence identity to SEQ ID NO: 45. In some aspects, the sequence of the variable light chain of sarilumab is shown in SEQ ID NO: 45.

In some aspects, the bispecific protein comprises a heavy chain immunoglobulin variable domain (VH) comprising an amino acid sequence having at least about 80%, at least about 85%, at least about 95%, at least about 96%, at least about 97%, at least about 98%, at least about 99%, or about 100% sequence identity to SEQ ID NO: 46. In some aspects, the sequence of the variable heavy chain of satralizumab is shown in SEQ ID NO: 46.

In some aspects, the bispecific protein comprises a light chain immunoglobulin variable domain (VL) comprising an amino acid sequence having at least about 80%, at least about 85%, at least about 95%, at least about 96%, at least about 97%, at least about 98%, at least about 99%, or about 100% sequence identity to SEQ ID NO: 47. In some aspects, the sequence of the variable light chain of satralizumab is shown in SEQ ID NO: 47.

In some aspects, the bispecific protein comprises a heavy chain immunoglobulin variable domain (VH) comprising an amino acid sequence having at least about 80%, at least about 85%, at least about 95%, at least about 96%, at least about 97%, at least about 98%, at least about 99%, or about 100% sequence identity to SEQ ID NO: 48. In some aspects, the sequence of the variable heavy chain of olokizumab is shown in SEQ ID NO: 48.

In some aspects, the bispecific protein comprises a light chain immunoglobulin variable domain (VL) comprising an amino acid sequence having at least about 80%, at least about 85%, at least about 95%, at least about 96%, at least about 97%, at least about 98%, at least about 99%, or about 100% sequence identity to SEQ ID NO: 49. In some aspects, the sequence of the variable light chain of olokizumab is shown in SEQ ID NO: 49.

In some aspects, the bispecific protein comprises an amino acid sequence having at least about 80%, at least about 85%, at least about 95%, at least about 96%, at least about 97%, at least about 98%, at least about 99%, or about 100% sequence identity to SEQ ID NO: 50. In some aspects, the sequence of vobarilizumab is shown in SEQ ID NO: 50.

### IIB(iii) CD40 Binding Domain

CD40 is a tumor necrosis factor (TNF) receptor superfamily member, mainly expressed on antigen-presenting cells such as B cells. CD40L (or CD154) is its ligand, which is expressed mostly on activated T cells. CD40-CD40L interactions have been shown to be important for B cell response and are implicated in human autoimmune diseases and immunodeficiency.

In some aspects, the CD40 binding molecule comprises an antibody that specifically binds to CD40 or an antigen binding portion thereof.

In some aspects, the anti-CD40 antibody or antigen binding portion thereof specifically binds to the same epitope as iscalimab. In some aspects, the anti-CD40 antibody or antigen binding portion thereof cross competes with iscalimab. Iscalimab can be found in PCT/EP2011/070058, published as WO2012065950A1 on May 24, 2012, which is incorporated herein by reference in its entirety.

In some aspects, the CD40 binding molecule (BD1) comprises a heavy chain immunoglobulin variable domain (VH) and a light chain immunoglobulin variable domain (VL), which bind to CD40, wherein:
(a) the VH comprises a VH-CDR1 sequence of GFTFSSYGMH (SEQ ID NO: 27), a VH-CDR2 sequence of VISYEESNRYHADSVKG (SEQ ID NO: 28), and a VH-CDR3 sequence of DGGIAAPGPDY (SEQ ID NO: 29), and
(b) the VL comprises a VL-CDR1 sequence of RSSQSLLYSNGYNYLD (SEQ ID NO: 30), a VL-CDR2 sequence of LGSNRAS (SEQ ID NO: 31), and a VL-CDR3 sequence of MQARQTPF (SEQ ID NO: 32).

In some aspects, the CD40 binding domain comprises a VH and a VL, wherein the VH comprises an amino acid sequence that has at least about 80%, at least about 85%, at least about 95%, at least about 96%, at least about 97%, at least about 98%, at least about 99%, or about 100% sequence identity to SEQ ID NO: 33; and/or the VL comprises an amino acid sequence that has at least about 80%, at least about 85%, at least about 95%, at least about 96%, at least about 97%, at least about 98%, at least about 99%, or about 100% sequence identity to SEQ ID NO: 34.

In some aspects, the CD40 binding domain comprises a heavy chain and a light chain, wherein the heavy chain is selected from the group consisting of the amino acid sequence as set forth in SEQ ID Nos: 11 to 18, or a variant thereof having one or more conservative amino acid substitutions, and the light chain comprises the amino acid sequence as set forth in SEQ ID No: 2, or a variant thereof having one or more conservative amino acid substitutions. In some aspects, the heavy chain comprises an amino acid sequence having at least about 95% sequence identity to the HC sequence as set forth in any one of SEQ ID NOs: 11 to 18; and/or the light chain comprises an amino acid sequence having at least about 95% sequence identity to the LC sequence as set forth in SEQ ID NO: 2.

The anti-CD40 antibodies useful for the bispecific protein are not limited to iscalimab. Other anti-CD40 antibodies that can be used to construct the bispecific protein include bleselumab, ravagalimab, lucatumumab, BMS-986325, teneliximab, BI-655064, and KPL-404. Bleselumab can be found in Okimura K, Maeta K, Kobayashi N, et al. Characterization of ASKP1240, a fully human antibody targeting human CD40 with potent immunosuppressive effects. Am J Transplant. 2014; 14(6): 1290-1299. doi: 10.1111/ajt. 12678, which is incorporated herein by reference in its entirety. Ravagalimab can be found in U.S. application Ser. No. 15/167,598, published as US 2016-0347850 A1 on Dec. 1, 2016, which is incorporated herein by reference in its entirety. Lucatumumab can be found in U.S. application Ser. No. 10/577,390, published as US20080057070A1 on Mar. 6, 2008, which is incorporated herein by reference in its entirety. BMS-986325 can be found in PCT/US2021/032815, published as WO2021236546A1 on Nov. 25, 2021. Teneliximab can be found at PCT/US1999/002949, published as WO1999042075A2 on Aug. 26, 1999, which is incorporated herein by reference in its entirety. BI-655064 can be found in U.S. application Ser. No. 13/075,303, published as US 2011-0243932 A1 on Oct. 6, 2011, which is incorporated herein by reference in its entirety. KPL-404 can be found in U.S. application Ser. No. 15/829,352, published as US 2018-0078640 A1 on Mar. 22, 2018, which is incorporated herein by reference in its entirety. In some aspects, the heavy chain and/or the light chain in the bispecific protein comprises an amino acid sequence having at least about 80%, at least about 85%, at least about 95%, at least about 96%, at least about 97%, at least about 98%, at least about 99%, or about 100% sequence identity to the sequence as set forth in any one of SEQ ID NOs: 51 to 66. Exemplary sequences of VH and VL of anti-CD40 antibodies useful for the bispecific protein are shown in SEQ ID NO: 51-66.

In some aspects, the bispecific protein comprises a heavy chain immunoglobulin variable domain (VH) comprising an amino acid sequence having at least about 80%, at least about 85%, at least about 95%, at least about 96%, at least about 97%, at least about 98%, at least about 99%, or about 100% sequence identity to SEQ ID NO: 51. In some aspects, the sequence of the variable heavy chain of iscalimab is shown in SEQ ID NO: 51.

In some aspects, the bispecific protein comprises a light chain immunoglobulin variable domain (VL) comprising an amino acid sequence having at least about 80%, at least about 85%, at least about 95%, at least about 96%, at least about 97%, at least about 98%, at least about 99%, or about 100% sequence identity to SEQ ID NO: 52. In some aspects, the sequence of the variable light chain of iscalimab is shown in SEQ ID NO: 52.

In some aspects, the bispecific protein comprises a heavy chain immunoglobulin variable domain (VH) comprising an amino acid sequence having at least about 80%, at least about 85%, at least about 95%, at least about 96%, at least about 97%, at least about 98%, at least about 99%, or about 100% sequence identity to SEQ ID NO: 53. In some aspects, the sequence of the variable heavy chain of bleselumab is shown in SEQ ID NO: 53.

In some aspects, the bispecific protein comprises a light chain immunoglobulin variable domain (VL) comprising an amino acid sequence having at least about 80%, at least about 85%, at least about 95%, at least about 96%, at least about 97%, at least about 98%, at least about 99%, or about 100% sequence identity to SEQ ID NO: 54. In some aspects, the sequence of the variable light chain of bleselumab is shown in SEQ ID NO: 54.

In some aspects, the bispecific protein comprises a heavy chain immunoglobulin variable domain (VH) comprising an amino acid sequence having at least about 80%, at least about 85%, at least about 95%, at least about 96%, at least about 97%, at least about 98%, at least about 99%, or about 100% sequence identity to SEQ ID NO: 55. In some aspects, the sequence of the variable heavy chain of ravagalimab is shown in SEQ ID NO: 55.

In some aspects, the bispecific protein comprises a light chain immunoglobulin variable domain (VL) comprising an amino acid sequence having at least about 80%, at least about 85%, at least about 95%, at least about 96%, at least about 97%, at least about 98%, at least about 99%, or about 100% sequence identity to SEQ ID NO: 56. In some aspects, the sequence of the variable light chain of ravagalimab is shown in SEQ ID NO: 56.

In some aspects, the bispecific protein comprises a heavy chain immunoglobulin variable domain (VH) comprising an amino acid sequence having at least about 80%, at least about 85%, at least about 95%, at least about 96%, at least about 97%, at least about 98%, at least about 99%, or about 100% sequence identity to SEQ ID NO: 57. In some aspects, the sequence of the variable heavy chain of lucatumumab is shown in SEQ ID NO: 57.

In some aspects, the bispecific protein comprises a light chain immunoglobulin variable domain (VL) comprising an amino acid sequence having at least about 80%, at least about 85%, at least about 95%, at least about 96%, at least about 97%, at least about 98%, at least about 99%, or about 100% sequence identity to SEQ ID NO: 58. In some aspects, the sequence of the variable light chain of lucatumumab is shown in SEQ ID NO: 58.

In some aspects, the bispecific protein comprises a heavy chain immunoglobulin variable domain (VH) comprising an amino acid sequence having at least about 80%, at least about 85%, at least about 95%, at least about 96%, at least about 97%, at least about 98%, at least about 99%, or about 100% sequence identity to SEQ ID NO: 59. In some aspects, the sequence of the variable heavy chain of BMS-986325 is shown in SEQ ID NO: 59.

In some aspects, the bispecific protein comprises a light chain immunoglobulin variable domain (VL) comprising an amino acid sequence having at least about 80%, at least about 85%, at least about 95%, at least about 96%, at least about 97%, at least about 98%, at least about 99%, or about 100% sequence identity to SEQ ID NO: 60. In some aspects, the sequence of the variable light chain of BMS-986325 is shown in SEQ ID NO: 60.

In some aspects, the bispecific protein comprises a heavy chain immunoglobulin variable domain (VH) comprising an amino acid sequence having at least about 80%, at least about 85%, at least about 95%, at least about 96%, at least about 97%, at least about 98%, at least about 99%, or about 100% sequence identity to SEQ ID NO: 61. In some aspects, the sequence of the variable heavy chain of teneliximab is shown in SEQ ID NO: 61.

In some aspects, the bispecific protein comprises a light chain immunoglobulin variable domain (VL) comprising an amino acid sequence having at least about 80%, at least about 85%, at least about 95%, at least about 96%, at least about 97%, at least about 98%, at least about 99%, or about 100% sequence identity to SEQ ID NO: 62. In some aspects, the sequence of the variable light chain of teneliximab is shown in SEQ ID NO: 62.

In some aspects, the bispecific protein comprises a heavy chain immunoglobulin variable domain (VH) comprising an amino acid sequence having at least about 80%, at least about 85%, at least about 95%, at least about 96%, at least about 97%, at least about 98%, at least about 99%, or about 100% sequence identity to SEQ ID NO: 63. In some aspects, the sequence of the variable heavy chain of BI-655064 is shown in SEQ ID NO: 63.

In some aspects, the bispecific protein comprises a light chain immunoglobulin variable domain (VL) comprising an amino acid sequence having at least about 80%, at least about 85%, at least about 95%, at least about 96%, at least about 97%, at least about 98%, at least about 99%, or about 100% sequence identity to SEQ ID NO: 64. In some aspects, the sequence of the variable light chain of BI-655064 is shown in SEQ ID NO: 64.

In some aspects, the bispecific protein comprises a heavy chain immunoglobulin variable domain (VH) comprising an amino acid sequence having at least about 80%, at least about 85%, at least about 95%, at least about 96%, at least about 97%, at least about 98%, at least about 99%, or about 100% sequence identity to SEQ ID NO: 65. In some aspects, the sequence of the variable heavy chain of KPL-404 is shown in SEQ ID NO: 65.

In some aspects, the bispecific protein comprises a light chain immunoglobulin variable domain (VL) comprising an amino acid sequence having at least about 80%, at least about 85%, at least about 95%, at least about 96%, at least about 97%, at least about 98%, at least about 99%, or about 100% sequence identity to SEQ ID NO: 66. In some aspects, the sequence of the variable light chain of KPL-404 is shown in SEQ ID NO: 66.

### IIC. Fc Region

In some aspects, the bispecific protein described herein further comprises an Fc region. As used herein, “Fc region” encompasses domains derived from the constant region of an immunoglobulin, preferably a human immunoglobulin, including a fragment, analog, variant, mutant or derivative of the constant region. Suitable immunoglobulins include IgG1, IgG2, IgG3, IgG4, and other classes such as IgA, IgD, IgE and IgM. The Fc region may be a native sequence Fc region or an altered Fc region. The Fc region of an immunoglobulin generally comprises two constant domains, a CH2 domain and a CH3 domain, and optionally comprises a CH4 domain. The bispecific protein of the disclosure includes an Fc region comprising a CH2 domain and a CH3 domain.

### IIC(i). Altered Fc Regions

Altered Fc regions (also referred to herein as “variant Fc regions”) can be used to alter the effector function and/or half-life of a bispecific protein of the disclosure. One or more alterations can be made in the Fc region in order to change functional and/or pharmacokinetic properties of molecules. Such alterations may result in a decrease or increase of C1q binding and complement dependent cytotoxicity (CDC) or of FcγR binding, for IgG, and antibody-dependent cellular cytotoxicity (ADCC), or antibody dependent cell mediated phagocytosis (ADCP). The present disclosure encompasses bispecific proteins wherein changes have been made to fine tune the effector function, either by enhancing or diminishing function or providing a desired effector function. Accordingly, in one aspect of the disclosure, the bispecific protein comprises a variant Fc region (i.e., Fc regions that have been altered as discussed below). Bispecific proteins comprising a variant Fc region are also referred to here as “Fc variants” As used herein “native” refers to the unmodified parental sequence and the bispecific protein comprising a native Fc region is herein referred to as a “native Fc bispecific protein”. Fc variants can be generated by numerous methods well known to one skilled in the art. Non-limiting examples include, isolating antibody coding regions (e.g., from hybridoma) and making one or more desired substitutions in the Fc region. Alternatively, the antigen-binding portion (e.g., variable regions) of a bispecific protein can be subcloned into a vector encoding a variant Fc region. In some aspects, the variant Fc region exhibits a similar level of inducing effector function as compared to the native Fc region. In some aspects, the variant Fc region exhibits a higher induction of effector function as compared to the native Fc. In some aspects, the variant Fc region exhibits lower induction of effector function as compared to the native Fc. Some specific aspects of variant Fc regions are detailed infra. Methods for measuring effector function are well known in the art.

In general, the effector function is modified through changes in the Fc region, including but not limited to, amino acid substitutions, amino acid additions, amino acid deletions and changes in post translational modifications to amino acids (e.g., glycosylation). The methods described below may be used to fine tune the effector function of a bispecific protein of the disclosure, or a ratio of the binding properties of the Fc region for FcRs (e.g., affinity and specificity), resulting in a bispecific protein with the desired properties.

It is understood that the Fc region as used herein includes the polypeptides comprising the constant region of an antibody molecule, excluding the first constant region immunoglobulin domain. Thus, Fc refers to the last two constant region immunoglobulin domains of IgA, IgD, and IgG, and the last three constant region immunoglobulin domains of IgE and IgM, and, optionally, all or a portion of the flexible hinge N-terminal to these domains. For IgA and IgM, Fc may include the J chain. For IgG, Fc comprises immunoglobulin domains Cgamma2 and Cgamma3 (Cγ2 and Cγ3) and optionally a portion of the lower hinge between Cgamma1 (Cγ1) and Cgamma2 (Cγ2). Although the boundaries of the Fc region may vary, as used herein the human IgG heavy chain Fc region comprises residues A231 to its carboxyl-terminus, wherein the numbering is according to the EU index as set forth in Kabat. Fc may refer to this region in isolation, or this region in the context of an antibody, antibody fragment, or Fc fusion protein. Polymorphisms have been observed at a number of different Fc positions, including but not limited to positions 270, 272, 312, 315, 356, and 358 of lgG1 as numbered by the EU index, and thus slight differences between the presented sequence and sequences in the prior art may exist.

In some aspects, the present disclosure encompasses Fc variants which have altered binding properties for a ligand (e.g., an Fc receptor, C1q) relative to a native Fc. Examples of binding properties include but are not limited to, binding specificity, equilibrium dissociation constant (K_{d}), dissociation and association rates (k_{off} and kₒₙ respectively), binding affinity and/or avidity. It is known in the art that the equilibrium dissociation constant (K_{d}) is defined as k_{off}/kₒₙ. In certain aspects, a bispecific protein comprising an Fc variant region with a low K_{d} may be more desirable than a bispecific protein with a high K_{d}. However, in some instances the value of the kₒₙ or k_{off} may be more relevant than the value of the K_{d}. One skilled in the art can determine which kinetic parameter is most important for a given application. For example, a modification that reduces binding to one or more positive regulator (e.g., FcγRIIIA) and/or enhanced binding to an inhibitory Fc receptor (e.g., FcγRIIB) would be suitable for reducing ADCC activity. Accordingly, the ratio of binding affinities (e.g., the ratio of equilibrium dissociation constants (K_{d}) for different receptors can indicate if the ADCC activity of an Fc variant in a bispecific protein of the disclosure is enhanced or decreased. Additionally, a modification that reduces binding to C1q would be suitable for reducing or eliminating CDC activity.

In some aspects, an Fc variant bispecific protein exhibits altered binding affinity for one or more Fc receptors including, but not limited to FcRn, FcγRI (CD64, including isoforms FcγRIA, FcγRIB, and FcγRIC; FcγRII (CD32, including isoforms FcγRIIA, FcγRIIB, and FcγRIIC); and FcγRIII (CD16, including isoforms FcγRIIIA and FcγRIIIB) as compared to a native Fc bispecific protein.

In certain aspects, an Fc variant bispecific protein has increased affinity for an Fc ligand. In other aspects, an Fc variant bispecific protein has decreased affinity for an Fc ligand relative to a native Fc bispecific protein.

It is contemplated that Fc variant bispecific proteins are characterized by in-vitro functional assays for determining one or more FcγR mediated effector cell functions. In certain aspects, Fc variant bispecific proteins have similar binding properties and effector cell functions in in-vivo models (such as those described and disclosed herein) as those in in-vitro based assays. However, the present disclosure does not exclude Fc variant bispecific proteins that do not exhibit the desired phenotype in in-vitro based assays but do exhibit the desired phenotype in vivo.

The serum half-life of proteins comprising Fc regions may be increased by increasing the binding affinity of the Fc region for FcRn. The term “antibody half-life” as used herein means a pharmacokinetic property of an antibody that is a measure of the mean survival time of antibody molecules following their administration. Antibody half-life can be expressed as the time required to eliminate 50% of a known quantity of immunoglobulin from the patient's body (or other mammal) or a specific compartment thereof, for example, as measured in serum, i.e., circulating half-life, or in other tissues. Half-life may vary from one immunoglobulin or class of immunoglobulin to another. In general, an increase in antibody (or bispecific protein) half-life results in an increase in mean residence time (MRT) in circulation for the bispecific protein administered.

The increase in half-life allows for the reduction in amount of drug given to a patient as well as reducing the frequency of administration. To increase the serum half-life of a bispecific protein, one may incorporate a salvage receptor binding epitope into the bispecific protein (especially an antibody fragment) as described in U.S. Pat. No. 5,739,277, for example. As used herein, the term “salvage receptor binding epitope” refers to an epitope of the Fc region of an IgG molecule (e.g., IgG1, IgG2, IgG3, or IgG4) that is responsible for increasing the in vivo serum half-life of the IgG molecule. Alternatively, bispecific proteins of the disclosure with increased half-lives may be generated by modifying amino acid residues identified as involved in the interaction between the Fc and the FcRn receptor (see, for examples, U.S. Pat. Nos. 6,821,505 and 7,083,784). In addition, the half-life of bispecific proteins of the disclosure may be increased by conjugation to PEG or albumin by techniques widely utilized in the art.

It is contemplated that either insertion of additional binding domains into the Fc region as described here and/or subsequent binding by antigen may affect Fc activity. For instance, binding antigen may increase or decrease binding affinity and activity for FcγRs, Clq, and FcRn. This would create an antigen-dependent switch to modulate various antibody-dependent processes. In some aspects, antigen binding may decrease interaction with FcRn, allowing a free bispecific protein to interact with FcRn and have a normal half-life, but allow rapid clearance/cellular internalization of bispecific protein-antigen complexes. Further, this could allow BD2-antigen mediated interactions to have an effect on the clearance of antigens bound by BD1, or vice versa.

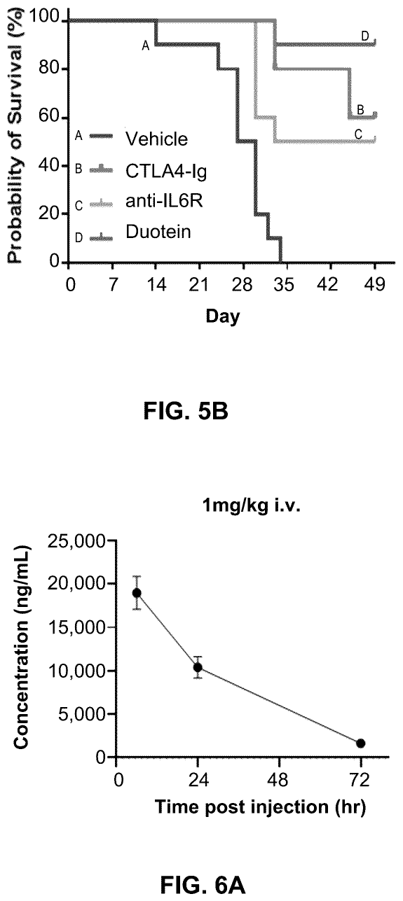

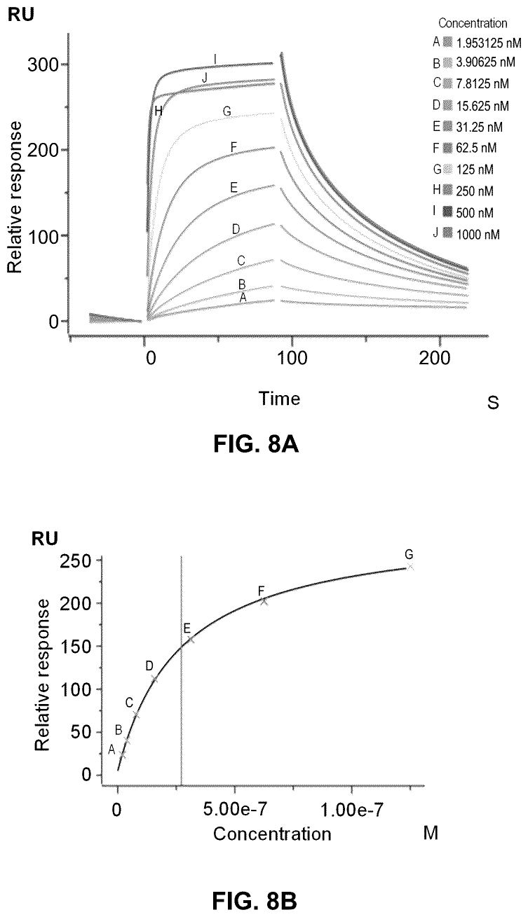

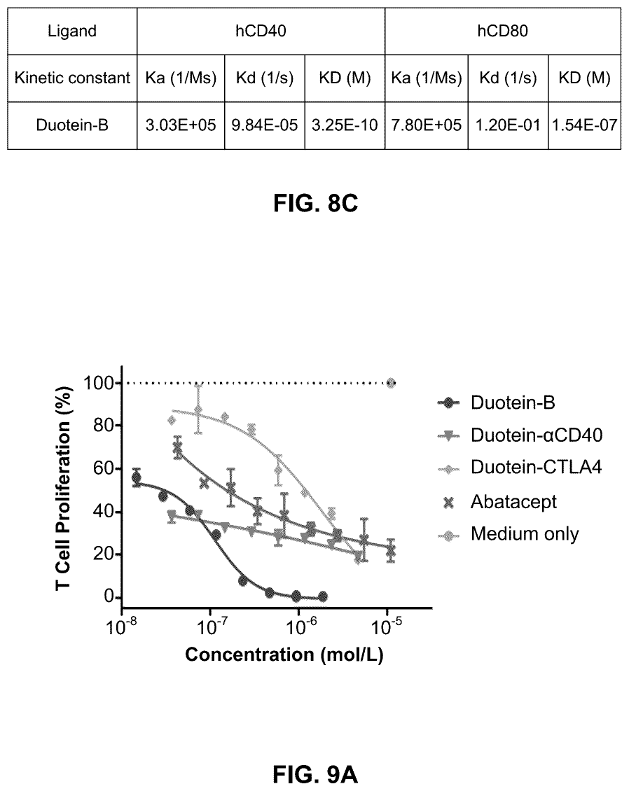

In some aspects, the present disclosure provides a bispecific protein comprising an Fc variant region, wherein the Fc region comprises a modification (e.g., amino acid substitutions, amino acid insertions, amino acid deletions) at one or more positions selected from the group consisting of 221, 225, 228, 234, 235, 236, 237, 238, 239, 240, 241, 243, 244, 245, 247, 250, 251, 252, 254, 255, 256, 257, 262, 263, 264, 265, 266, 267, 268, 269, 279, 280, 284, 292, 296, 297, 298, 299, 305, 308, 313, 316, 318, 320, 322, 325, 326, 327, 328, 329, 330, 331, 332, 333, 334, 339, 341, 343, 370, 373, 378, 392, 416, 419, 421, 428, 433, 434, 435, 436, 440, and 443 as numbered by the EU index as set forth in Kabat. Optionally, the Fc region may comprise a modification at additional and/or alternative positions known to one skilled in the art (see, e.g., U.S. Pat. Nos. 5,624,821; 6,277,375; 6,737,056; 7,083,784; 7,317,091; 7,217,797; 7,276,585; 7,355,008). Additional, useful amino acid positions and specific substitutions are exemplified in Tables 2, and 6-10 of U.S. Pat. No. 6,737,056; the tables presented in FIG. 41 of US 2006/024298; the tables presented in FIGS. 5, 12, and 15 of US 2006/235208; the tables presented in FIGS. 8, 9 and 10 of US 2006/0173 170 and the tables presented in FIGS. 8-10, 13 and 14 of WO 09/058492.

In some aspects, the present disclosure provides a bispecific protein comprising an Fc variant region, wherein the Fc region comprises at least one substitution selected from the group consisting of 221K, 221Y, 225E, 225K, 225W, 228P, 234D, 234E, 234N, 234Q, 234T, 234H, 234Y, 2341, 234V, 234F, 235A, 235D, 235R, 235W, 235P, 235S, 235N, 235Q, 235T, 235H, 235Y, 2351, 235V, 235E, 235F, 236E, 237L, 237M, 237P, 239D, 239E, 239N, 239Q, 239F, 239T, 239H, 239Y, 2401, 240A, 240T, 240M, 241W, 241L, 241Y, 241E, 241R, 243W, 243L, 243Y, 243R, 243Q, 244H, 245A, 247L, 247V, 247G, 250E, 250Q, 251F, 252L, 252Y, 254S, 254T, 255L, 256E, 256F, 256M, 257C, 257M, 257N, 2621, 262A, 262T, 262E, 2631, 263A, 263T, 263M, 264L, 264I, 264W, 264T, 264R, 264F, 264M, 264Y, 264E, 265A, 265G, 265N, 265Q, 265Y, 265F, 265V, 2651, 265L, 265H, 265T, 2661, 266A, 266T, 266M, 267Q, 267L, 268E, 269H, 269Y, 269F, 269R, 270E, 280A, 284M, 292P, 292L, 296E, 296Q, 296D, 296N, 296S, 296T, 296L, 2961, 296H, 296G, 297S, 297D, 297E, 298A, 298H, 298I, 298T, 298F, 2991, 299L, 299A, 299S, 299V, 299H, 299F, 299E, 3051, 308F, 313F, 316D, 318A, 318S, 320A, 320S, 322A, 322S, 325Q, 325L, 3251, 325D, 325E, 325A, 325T, 325V, 325H, 326A, 326D, 326E, 326G, 326D, 326V, 327G, 327W, 327N, 327L, 328S, 328M, 328D, 328E, 328N, 328Q, 328F, 328I, 328V, 328T, 328H, 328A, 329F, 329H, 329Q, 330K, 330G, 330T, 330C, 330L, 330Y, 330V, 3301, 330F, 330R, 330H, 331G, 331A, 331L, 331M, 331F, 331W, 331K, 331Q, 331E, 331S, 331V, 3311, 331C, 331Y, 331H, 331R, 331N, 331D, 331T, 332D, 332S, 332W, 332F, 332E, 332N, 332Q, 332T, 332H, 332Y, 332A, 333A, 333D, 333G, 333Q, 333S, 333V, 334A, 334E, 334H, 334L, 334M, 334Q, 334V, 334Y, 339T, 370E, 370N, 378D, 392T, 396L, 416G, 419H, 421K, 428L, 428F, 433K, 433L, 434A, 424F, 434W, 434Y, 436H, 440Y and 443W as numbered by the EU index as set forth in Kabat. Optionally, the Fc region can further comprise additional and/or alternative amino acid substitutions known to one skilled in the art including, but not limited to, those exemplified in Tables 2, and 6-10 of U.S. Pat. No. 6,737,056; the tables presented in FIG. 41 of US 2006/024298; the tables presented in FIGS. 5, 12, and 15 of US 2006/235208; the tables presented in FIGS. 8, 9 and 10 of US 2006/0173170 and the tables presented in FIGS. 8, 9 and 10 of US20090041770, all of which are incorporated herein by reference.

In some aspects, the disclosure provides a bispecific protein comprising an Fc variant region, wherein the Fc region comprises at least one modification (e.g., amino acid substitutions, amino acid insertions, amino acid deletions) at one or more positions selected from the group consisting of 228, 234, 235 and 331 as numbered by the EU index as set forth in Kabat. In some aspects, the modification is at least one substitution selected from the group consisting of 228P, 234F, 235E, 235F, 235Y, and 331S as numbered by the EU index as set forth in Kabat.

In some aspects, the present disclosure provides a bispecific protein comprising an Fc variant region, wherein the Fc region is an IgG4 Fc region and comprises at least one modification at one or more positions selected from the group consisting of 228 and 235 as numbered by the EU index as set forth in Kabat. In some aspects, the Fc region is an IgG4 Fc region and the non-naturally occurring amino acids are selected from the group consisting of 228P, 235E and 235Y as numbered by the EU index as set forth in Kabat.

In some aspects, the present disclosure provides a bispecific protein comprising an Fc variant region, wherein the Fc region comprises at least one non-naturally occurring amino acid at one or more positions selected from the group consisting of 239, 330 and 332 as numbered by the EU index as set forth in Kabat. In some aspects, the modification is at least one substitution selected from the group consisting of 239D, 330L, 330Y, and 332E as numbered by the EU index as set forth in Kabat. See, U.S. Pat. No. 7,317,091, incorporated herein by referenced in its entirety.

In some aspects, the present disclosure provides a bispecific protein comprising an Fc variant region, wherein the Fc region comprises at least one non-naturally occurring amino acid at one or more positions selected from the group consisting of 252, 254, and 256 as numbered by the EU index as set forth in Kabat. In some aspects, the modification is at least one substitution selected from the group consisting of 252Y, 254T and 256E as numbered by the EU index as set forth in Kabat. See, U.S. Pat. No. 7,083,784, incorporated herein by reference in its entirety.

In some aspects, the present disclosure provides a bispecific protein comprising an Fc variant region, wherein the Fc region comprises a non-naturally occurring amino acid at position 428 as numbered by the EU index as set forth in Kabat. In some aspects, the modification at position 428 is selected from the group consisting of 428T, 428L, 428F, and 428S as numbered by the EU index as set forth in Kabat. See, U.S. Pat. No. 7,670,600, incorporated herein by reference in its entirety. In some aspects, an Fc variant may further comprise a non-naturally occurring amino acid at position 434 as numbered by the EU index as set forth in Kabat. In some aspects, the modification at position 434 is selected from the group consisting of 434A, 434S, and 434F as numbered by the EU index as set forth in Kabat. In other aspects, the present disclosure provides a bispecific protein comprising an Fc variant region, wherein the Fc region comprises a non-naturally occurring amino acid at positions 428 and 434 as numbered by the EU index as set forth in Kabat. In some aspects, the Fc region comprises 428L, 434S. See, U.S. Pat. No. 8,088,376.

In some aspects, the effector functions elicited by IgG antibodies strongly depend on the carbohydrate moiety linked to the Fc region of the protein (Claudia Ferrara et al., 2006, Biotechnology and Bioengineering 93:851-861). Thus, glycosylation of the Fc region can be modified to increase or decrease effector function (see for examples, Umana et al, 1999, Nat. Biotechnol17:176-180; Davies et al., 2001, Biotechnol Bioeng 74:288-294; Shields et al, 2002, J Biol Chern 277:26733-26740; Shinkawa et al., 2003, J Biol Chern 278:3466-3473; U.S. Pat. Nos. 6,602,684; 6,946,292; 7,064,191; 7,214,775; 7, 393,683; 7,425,446; 7,504,256; POTELLIGENT™ technology (Biowa, Inc. Princeton, N.J.); GLYCOMAB™ glycosylation engineering technology (GLYCART biotechnology AG, Zurich, Switzerland)). Accordingly, in some aspects the Fc regions of bispecific proteins disclosed herein comprise altered glycosylation of amino acid residues. In some aspects, the altered glycosylation of the amino acid residues results in lowered effector function. In some aspects, the altered glycosylation of the amino acid residues results in increased effector function. In some aspects, the Fc region has reduced fucosylation. In some aspects, the Fc region is afucosylated (see for examples, U.S. Patent Application Publication No. 2005/0226867). In some aspects, these bispecific proteins with increased effector function, specifically ADCC, are generated in host cells (e.g., CHO cells, Lemna minor) engineered to produce highly defucosylated polypeptide with over 100-fold higher ADCC compared to polypeptide produced by the parental cells (Mori et al., 2004, Biotechnol Bioeng 88:901-908; Cox et al., 2006, Nat Biotechnol., 24: 1591-7).

Addition of sialic acid to the oligosaccharides on IgG molecules can enhance their anti-inflammatory activity and alter their cytotoxicity (Keneko et al., Science, 2006, 313:670-673; Scallon et al., Mol. Immuno. 2007 March; 44(7): 1524-34). The studies referenced above demonstrate that IgG molecules with increased sialylation have anti-inflammatory properties whereas IgG molecules with reduced sialylation have increased immunostimulatory properties (e.g., increase ADCC activity). Therefore, a bispecific protein can be modified with an appropriate sialylation profile for a particular application (US Publication No. 2009/0004179 and International Publication No. WO 2007/005786).

In some aspects, the Fc regions of bispecific proteins disclosed comprise an altered sialylation profile compared to the native Fc region. In some aspects, the Fc regions of bispecific proteins disclosed herein comprise an increased sialylation profile compared to the native Fc region. In some aspects, the Fc regions of bispecific proteins disclosed herein comprise a decreased sialylation profile compared to the native Fc region.

In some aspects, the Fc variants useful for the present disclosure can be combined with other known Fc variants such as those disclosed in Ghetie et al., 1997, Nat Biotech. 15:637-40; Duncan et al, 1988, Nature 332:563-564; Lund et al., 1991, J. Immunol147:2657-2662; Lund et al, 1992, Mol Immunol29:53-59; Alegre et al, 1994, Transplantation 57:1537-1543; Hutchins et al., 1995, Proc Natl. Acad Sci US A 92:11980-11984; Jefferis et al, 1995, Immunol Lett. 44:111-117; Lund et al., 1995, Faseb J 9:115-119; Jefferis et al, 1996, Immunol Lett 54:101-104; Lund et al, 1996, J Immunol 157:4963-4969; Armour et al., 1999, Eur J Immunol29:2613-2624; Idusogie et al, 2000, J Immunol164:4178-4184; Reddy et al, 2000, J Immunol164: 1925-1933; Xu et al., 2000, Cell Immunol200:16-26; Idusogie et al, 2001, J Immunol166:2571-2575; Shields et al., 2001, J Biol Chern 276:6591-6604; Jefferis et al, 2002, Immunol Lett 82:57-65; Presta et al., 2002, Biochem Soc Trans 30:487-490); U.S. Pat. Nos. 5,624,821; 5,885,573; 5,677,425; 6,165,745; 6,277,375; 5,869,046; 6,121,022; 5,624,821; 5,648,260; 6,528,624; 6,194,551; 6,737,056; 7,122,637; 7,183,387; 7,332,581; 7,335,742; 7,371,826; 6,821,505; 6,180,377; 7,317,091; 7,355,008. Other modifications and/or substitutions and/or additions and/or deletions of the Fc domain will be readily apparent to one skilled in the art.

It is notable that polypeptides presented in the bispecific protein format comprising a native Fc retain the ability to bind FcRn and C1q and to mediate ADCC, as shown in the examples. Thus, in some aspects, a bispecific protein retains the ability to bind FcRn and/or C1q and/or one or more Fc gamma receptors (FcγRs). For example, in some aspects, a bispecific protein retains at least 70%, 75%, 80%, 85%, 90%, 95%, 96%, 97%, 98%, 99%, or 100% of the ability to bind FcRn and/or C1q and/or one or more FcγRs, as compared to a conventional antibody that binds to one of the epitopes to which the bispecific protein binds. In some aspects, a bispecific protein is generated from the binding domains of one or two conventional antibodies, and the comparison of activity is made to one or both of those conventional antibodies.

Altered Fc regions may also be used to generate heavy chain heterodimers, resulting in bispecific proteins comprising two different heavy-light chain pairs. To facilitate the formation of heterodimers the interface between a pair of Fc regions is engineered to maximize the percentage of heterodimers which are recovered from recombinant cell culture.

In some aspects, the interface comprises at least a part of the CH3 domain. In this method, a “protrusion” is generated by replacing one or more, small amino acid side chains from the interface of the first antibody molecule with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers. CH3 modifications include, for example, Y 407V/T366S/L368A on one heavy chain and T366W on the other heavy chain; S354C/T366W on one heavy chain and Y349C/Y 407V/T366S/L368A on the other heavy chain. Additional modifications resulting in a protrusion on one chain and a cavity on the other are described in U.S. Pat. No. 7,183,076; US 2014/0348839; and Merchant et al., 1998, Nat. Biotech 16:677-681. Some non-limiting examples of modifications that can result in a protrusion-cavity arrangement are presented in Table 2. Other modifications which may be used to generate heterodimers include but are not limited to those which alter the charge polarity across the Fc dimer interface such that co-expression of electrostatically matched Fc regions results in heterodimerization. Modifications which alter the charge polarity include, but are not limited to, those presented in Table 3 below (also see, US20090182127; Gunasekaran et al., 2010, JBC 285:19637-46). In addition, Davis et al. (20 10, Prot. Eng. Design & Selection 23: 195-202) describe a heterodimeric Fc platform using strand-exchanged engineered domain (SEED) CH3 regions which are derivatives of human IgG and IgA CH3 domains (also, see WO 2007/110205).

TABLE 2
CH3 modifications for heterodimerization (protrusion-cavity)

| Modification(s) in one heavy chain | Modification(s) in other heavy chain |
|---|---|
| T366Y | Y407T |
| T366W | Y407A |
| T366Y | Y407T |
| T394W | F405A |
| T366Y/F405A | T394W/Y407T |
| T366W/F405W | T394S/Y407A |
| F405W | T394S |
| D399C | K392C |
| T366W | T366S/L368A/Y407V |
| T366W/D399C | T366S/L368A/K392C/Y407V |
| T366W/K392C | T366S/D399C/L368A/Y407V |
| S354C/T366W | Y349C/T366S//L368A/Y407V |
| Y349C/T366W | S354C/T366S//L368A/Y407V |
| E356C/T366W | Y349C/T366S//L368A/Y407V |
| Y349C/T366W | E356C/T366S//L368A/Y407V |
| E357C/T366W | Y349C/T366S//L368A/Y407V |
| Y349C/T366W | E357C/T366S//L368A/Y407V |

TABLE 3
CH3 modifications for heterodimerization

| Modification(s) in one heavy chain | Modification(s) in other heavy chain |
|---|---|
| K370E/D399K/K439D | D356K/E357K/K409D |
| K409D | D399K |
| K409E | D399K |
| K409E | D399R |
| K409D | D399R |
| D339K | E356K |
| D399K/E356K | K409D/K392D |
| D399K/E356K | K409D/K439D |
| D399K/E357K | K409D/K370D |
| D399K/E356K/E357K | K409D/K392D/K370D |
| D399K/E357K | K409D/K392D |
| K392D/K409D | D399K |
| K409D/K360D | D399K |

A person skilled in the art would understand that in some aspects, an Fc fusion protein, including the bispecific protein disclosed herein, can form dimers due to the homodimeric nature of molecules comprising an Fc region. In some aspects the Fc regions of a bispecific protein may be differentially engineered with mutations to promote and/or maintain heterodimerization (e.g., chimeric mutations, complementary mutations, dock and lock mutations, knobs into holes mutations, strand-exchange engineered domain (SEED) mutations, etc., see for example, U.S. Pat. No. 7,183,076; Merchant et al. (1998) Nat. Biotech 16:677-681; Ridgway et al. (1996) Protein Engineering 9:617-621; Davis et al. (201 0) Prot. Eng. Design & Selection 23:195-202; WO 2007/110205; WO 2007/147901; Gunasekaran et al. (2010) JBC 285:19637-46, all incorporated herein by reference). Accordingly, a bispecific protein can be engineered to form a heterodimer comprising for example a first binding domain fused to a first Fc region or fragment thereof, and a second (i.e., different) binding domain fused to a second Fc region or fragment, wherein the first and second Fc regions, or fragments thereof have been engineered to heterodimerize.

### IID. Glycosylation

In addition to the ability of glycosylation to alter the effector function of polypeptides, modified glycosylation in the variable region can alter the affinity of the antibody (or bispecific proteins) for a target antigen. In one aspect, the glycosylation pattern in the variable region of the present bispecific protein is modified. For example, an aglycosylated bispecific protein (i.e., the bispecific protein lacks glycosylation) can be made. Glycosylation can be altered to, for example, increase the affinity of the bispecific protein for a target antigen. Such carbohydrate modifications can be accomplished by, for example, altering one or more sites of glycosylation within the bispecific protein sequence. For example, one or more amino acid substitutions can be made that result in elimination of one or more variable region framework glycosylation sites to thereby eliminate glycosylation at that site. Such aglycosylation may increase the affinity of the bispecific protein for antigen. Such an approach is described in further detail in U.S. Pat. Nos. 5,714,350 and 6,350,861. One or more amino acid substitutions can also be made that result in elimination of a glycosylation site present in the Fc region (e.g., Asparagine 297 of lgG). Furthermore, aglycosylated bispecific proteins may be produced in bacterial cells which lack the necessary glycosylation machinery.

### IIE. Polypeptide Linkers

Linkers may be used to join domains/regions of the bispecific protein into a contiguous molecule. As described herein, a bispecific protein may include one, two, or more linker polypeptides, (e.g., L1 and L2). Additionally, a bispecific protein may include additional linkers, such as a flexible linker interconnecting the variable heavy and light chains of an scFv and other linkers that connect other binding units to the core structure.

An exemplary, non-limiting example of a linker is a polypeptide chain comprising at least 4 residues. Portions of such linkers may be flexible, hydrophilic and have little or no secondary structure of their own (linker portions or flexible linker portions). Linkers of at least 4 amino acids may be used to join domains and/or regions that are positioned near to one another after the molecule has assembled. Longer or shorter linkers may also be used. Thus, linkers may be approximately 1, 2, 3, 4, 5, 6, 7, 8, 9, 10, 11, 12, 13, 14, 15, 16, 17, 18, 19, 20, 25, 30, 35, 40, 45, or approximately 50 residues in length. When multiple linkers are used to interconnect portions of the molecule, the linkers may be the same or different (e.g., the same or different length and/or amino acid sequence).

The linker(s) facilitate formation of the desired structure. Linkers may comprise (Gly-Ser)n residues, with some Glu or Lys residues dispersed throughout to increase solubility. Alternatively or additionally, linkers may not comprise any Serine residues, such linkers may be preferable where the linker is subject to O-linked glycosylation. In some aspects, linkers may contain cysteine residues, for example, if dimerization of linkers is used to bring the domains of the bispecific protein into their properly folded configuration. In some aspects, the bispecific protein comprises at least two polypeptide linkers that join domains of the polypeptide. In other aspects, the bispecific protein comprises at least three polypeptide linkers. In other aspects the bispecific protein comprises four or more polypeptide linkers.

In some aspects, the polypeptide linker comprises a portion of an Fc moiety. For example, in some aspects, the polypeptide linker can comprise a portion of immunoglobulin hinge domain of an IgG1, IgG2, IgG3, and/or IgG4 antibody. In some aspects, the polypeptide linker comprises a portion of a mutated immunoglobulin hinge domain of an IgG1, IgG2, IgG3 and/or IgG4. In some aspects, the polypeptide linker comprises at least 5, 7, 8, or 15 amino acid residues of an immunoglobulin hinge region/domain of an IgG1, IgG2, IgG3, and/or IgG4 antibody. In some aspects, the polypeptide linker comprises at least 5, 7, 8, or 15 amino acid residues of a modified immunoglobulin hinge region/domain of an IgG1, IgG2, IgG3, and/or IgG4 antibody.

The polypeptide linker may comprise all, or a portion of a hinge region that naturally comprises three cysteines. In certain aspects, the selected hinge region is truncated or otherwise altered or substituted relative to the complete and/or naturally-occurring hinge region such that only one or two of the cysteine residues remain. Similarly, in certain other aspects, the polypeptide linker may comprise a mutated or otherwise altered portion of a hinge region in which the number of cysteine residues is reduced by amino acid substitution or deletion, for example a mutated or otherwise altered hinge region containing zero, one or two cysteine residues as described herein. A mutated or otherwise altered hinge domain may thus be derived or constructed from (or using) a wild-type immunoglobulin hinge domain that contains one or more cysteine residues. In certain aspects, a mutated or otherwise altered portion of a hinge region may contain zero or only one cysteine residue, wherein the mutated or otherwise altered hinge region is or has been derived from a wild type immunoglobulin hinge region that contains, respectively, one or more or two or more cysteine residues. In the mutated or otherwise altered portion of a hinge region, the cysteine residues of the wild-type immunoglobulin hinge region are preferably deleted or substituted with amino acids that are incapable of forming a disulfide bond. In some aspects, a mutated or otherwise altered portion of a hinge region is or has been derived from a human IgG wild-type hinge region, which may include any of the four human IgG isotype subclasses, IgG1, IgG2, IgG3 or IgG4.

In some aspects, the polypeptide linker comprises a portion of a hinge region comprising the cysteine residue that forms a disulfide bond with an immunoglobulin light chain (EU residue 220). In some aspects, the polypeptide linker comprises an altered portion of a hinge region comprising an amino acid substitution at EU residue C220. In some aspects, the polypeptide linker comprises the amino acid substitution C220V.

In some aspects, the polypeptide linker comprises an amino acid substitution that prevents hinge-related spontaneous self-cleavage. In some aspects, the polypeptide linker comprises an amino acid substitution at position at EU position D221. In some aspects, the polypeptide linker comprises the amino acid substitution D221G. In some aspects, the polypeptide linker lacks the amino acid D221.

As discussed above, some aspects include one or more polypeptide linkers that comprise or consist of a gly-ser linker. As used herein, the term “gly-ser linker” refers to a peptide that consists of glycine and serine residues. An exemplary gly-ser linker comprises an amino acid sequence of the formula (Gly4Ser)ₙ or (Gly4Ser)ₙG, wherein n is a positive integer (e.g., 1, 2, 3, 4, 5, 6, 7, 8, 9 or 10), encompassing. Some used and non-limiting examples of a gly-ser linker includes (Gly₄Ser)₃, (Gly₄Ser)₄, (Gly₄Ser)₃G, or (Gly₄Ser)₄G. In yet other aspects, two or more gly-ser linkers are incorporated in series in a polypeptide linker. In some aspects, there is no linker sequences.

It is contemplated here that varying the length of the linkers flanking BD2 can impact on the orientation of the BD2 antigen binding site and spacing relative to the rest of the bispecific protein molecule. For example, a short N-terminal linker and long C-terminal linker may create an orientation where the binding site is conformed in one direction, while a long N-terminal and short C-terminal linker may impart an opposite conformational orientation. Accordingly, linker length may be modulated in order to orient the BD2 antigen binding site and have important impacts on creating or avoiding steric effects between BD1 and BD2 and/or BD2 and other entities that bind the antibody molecule in the Fc or other domains. Therefore, The length and amino acid sequence of a flexible linker connecting BD2 to the rest part of the bispecific protein may be selected and optimized (e.g., (Gly4Ser)ₙ, where n is 2, 3, or 4 or more).

### III. Labels, Conjugates and Moieties

In some features, drugs and other molecules may be targeted to a bispecific protein via site-specific conjugation. For example, bispecific proteins can comprise cysteine engineered domains (including cysteine(s) engineered into a binding unit and/or Fc domain), which result in free thiol groups for conjugation reactions. In some aspects, a bispecific protein is engineered to incorporate specific conjugation sites. In some aspects, the present disclosure provides a bispecific protein comprising an Fc variant region, wherein the Fc region comprises an amino acid substitution at one or more of positions 239, 282, 289, 297, 312, 324, 330, 335, 337, 339, 356, 359, 361, 383, 384, 398, 400, 440, 422, and 442, as numbered by the EU index. In some aspects, the Fc region comprises substitutions at one or more of the following groups of positions: a) 289 and 440; b) 330 and 440; c) 339 and 440; d) 359 and 440; e) 289 and 359; f) 330 and 359; g) 339 and 359; h) 289 and 339; i) 330 and 339; j) 289 and 330; k) 339 and 442; 1) 289, 339, and 442; m) 289, 330, and 339; n) 330, 339, and 442; and o) 289, 330, and 442. In other aspects, the present disclosure provides a bispecific protein, wherein the CH1 domain of the Fab arm comprises a substitution at one or more of positions 131, 132, 134, 135, 136 and 139, as numbered by the EU index. In some aspects, the substitution comprises a substitution to an amino acid chosen from cysteine, lysine, tyrosine, histidine, selenocysteine, and selenomethionine. In some aspects, the substitution is a cysteine. Methods for generating stable cysteine engineered antibodies are described in U.S. Pat. No. 7,855,275, U.S. 20110033378 and US20120213705, the contents of which are incorporated herein by reference in their entirety.

### IV. Nucleic Acid Molecules Encoding Bispecific Proteins

The present disclosure provides nucleic acid molecules that encode bispecific proteins. One aspect of the disclosure provides nucleic acid molecules encoding any of the bispecific proteins of the disclosure. A nucleic acid molecule may encode a heavy chain and/or light chain of any of the bispecific proteins that are disclosed herein, as well as any of the individual binding domains that are disclosed herein. One of skill in the art will appreciate that such polynucleotide molecules may vary in nucleotide sequence given nucleic acid codon degeneracy as well as codon frequency for particular organisms, as is generally known in the art.

In some aspects, the disclosure provides a vector comprising the nucleic acid molecule(s), a host cell comprising the vector or that is capable of producing a bispecific protein, or any modifications thereof.

In some aspects, the disclosure relates to methods for producing bispecific proteins. In some aspects, recombinant nucleic acid molecules that encode all or a portion of the bispecific protein disclosed herein are operably linked to one or more regulatory nucleotide sequences in an expression construct. The nucleic acid sequences encoding the light and chimeric heavy chains of the bispecific protein can be cloned in the same expression vector in any orientation (e.g., light chain in front of the heavy chain or vice versa) or can be cloned in two different vectors. If expression is carried out using one vector, the two coding genes can have their own genetic elements (e.g., promoter, RBS, leader, stop, poly A, etc) or they can be cloned with one single set of genetic elements, but connected with a cistron element. Regulatory nucleotide sequences will generally be appropriate for a host cell used for expression. Numerous types of appropriate expression vectors and suitable regulatory sequences are known in the art for a variety of host cells. Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the disclosure. The promoters may be either naturally occurring promoters, or hybrid promoters that combine elements of more than one promoter. An expression construct may be present in a cell on an episome, such as a plasmid, or the expression construct may be inserted in a chromosome.

In some aspects, the expression vector contains a selectable marker gene to allow the selection of transformed host cells. Selectable marker genes are well known in the art and will vary with the host cell used. In some aspects, this disclosure relates to an expression vector comprising a nucleotide sequence encoding a polypeptide and operably linked to at least one regulatory sequence. Regulatory sequences are art-recognized and are selected to direct expression of the encoded polypeptide. Accordingly, the term regulatory sequence includes promoters, enhancers, and other expression control elements. Exemplary, non-limiting regulatory sequences are described in Goeddel; Gene Expression Technology: Methods in Enzymology, Academic Press, San Diego, CA (1990). It should be understood that the design of the expression vector can depend on such factors as the choice of the host cell to be transformed and/or the type of protein desired to be expressed. Moreover, the copy number of the particular vector, the ability to control that copy number and the expression of any other protein encoded by the vector, such as antibiotic markers, should also be considered.

The disclosure further pertains to methods of producing a bispecific protein of the disclosure. For example, a host cell transfected with one or more than one expression vector encoding a bispecific protein (e.g., a single vector encoding the chimeric heavy and the light chain or two vectors, one encoding the chimeric heavy chain and one encoding the light chain) can be cultured under appropriate conditions to allow expression of the polypeptide to occur. The bispecific protein may be secreted and isolated from a mixture of cells and medium containing the polypeptide. Alternatively, the bispecific protein may be retained in the cytoplasm or in a membrane fraction and the cells harvested, lysed and the protein isolated. A cell culture includes host cells, media and other byproducts. Suitable media for cell culture are well known in the art. The bispecific protein can be isolated from cell culture medium, host cells, or both using techniques known in the art for purifying proteins, including ion-exchange chromatography, gel filtration chromatography, ultrafiltration, electrophoresis, and immunoaffinity purification. In some aspects, the bispecific protein is made as a bispecific protein containing a domain which facilitates its purification.

A recombinant nucleic acid can be produced by ligating the cloned gene, or a portion thereof, into a vector suitable for expression in either prokaryotic cells, eukaryotic cells (yeast, avian, insect or mammalian), or both. Expression vehicles for production of a recombinant polypeptide include plasmids and other vectors. For instance, suitable vectors include plasmids of the types: pBR322-derived plasmids, pEMBL-derived plasmids, pEXderived plasmids, pBTac-derived plasmids and pUC-derived plasmids for expression in prokaryotic cells, such as E. coli. In some aspects, mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pKO-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. The various methods employed in the preparation of the plasmids and in the transformation of host organisms are known in the art. For other suitable expression systems for both prokaryotic and eukaryotic cells, as well as general recombinant procedures, see Molecular Cloning A Laboratory Manual, 2^{nd} Ed., ed. by Sambrook, Fritsch and Maniatis (Cold Spring Harbor Laboratory Press, 1989) Chapters 16 and 17. In some instances, it may be desirable to express the recombinant polypeptide by the use of a baculovirus expression system Examples of such baculovirus expression systems include pVL-derived vectors (such as pVL1392, pVL1393 and pVL941), pAcUW-derived vectors (such as pAcUW1), and pBlueBac-derived vectors (such as the fi-gal containing pBlueBac III).

Once a molecule has been produced, it may be purified by any method known in the art for purification of a protein, an immunoglobulin molecule or other multimeric molecules using techniques such as, for example, chromatography (e.g., ion exchange, affinity, particularly by affinity for the specific antigens Protein A or Protein G, and sizing column chromatography), centrifugation, differential solubility, or by any other standard technique for the purification of proteins. Further, the molecules disclosed herein may be fused to heterologous polypeptide sequences (e.g., affinity tags) as are routinely employed to facilitate purification.

Regardless of how a bispecific protein is generated and purified, binding assays, for example, dual ELISA assays, may be performed (before and/or after purification) to confirm functional binding activity of the bispecific protein. Such binding assays are generally known in the art.

### V. Pharmaceutical Formulations and Therapeutic Uses

In some aspects, the disclosure provides pharmaceutical compositions. Such pharmaceutical compositions may be compositions comprising a nucleic acid molecule that encodes a bispecific protein. Such pharmaceutical compositions may also be compositions comprising a bispecific protein, a combination of bispecific proteins, or a combination of bispecific proteins and pharmaceutically acceptable excipients.

As discussed herein the bispecific proteins may be used to modulate the immune activities in a certain way as therapeutics to treat immunological disorders. For example, many immunological disorders are caused by abnormally activated immune responses against self-tissues, often involving multiple pathogenic pathways. In some aspects, the bispecific proteins disclosed herein binds to two different targets that each contributes to the abnormally activated immune responses through a distinct pathway. The bindings result in inhibition of both pathways at the same time that effectively prevents the pathogenic immune responses and related tissue damages.

In some aspects, the bispecific proteins disclosed herein binds to two different, but adjacently located epitopes. These bispecific proteins can exhibit markedly increased affinity (or avidity) and target residence time when both binding motifs bind simultaneously to their target sites. Compared to each binding motif alone, the bispecific proteins can achieve a high target selectivity and long duration of action, which are favorable property for therapeutical immunomodulators.

In some aspects, the bispecific proteins have one motif with immunomodulatory property connected to a second motif that binds to a protein expressed on the surface of certain cell types. This approach describe herein can deliver an immunomodulator specifically to certain cell types to achieve higher potency within those cells and to avoid undesired effects on the bystander cells. These bispecific proteins have increased specificity for desired biological effects and an improved safety profile.

Accordingly, provided herein are method of treating a disease or disorder a subject in need thereof, comprising administering to the subject a bispecific protein described herein such that the immune response in the subject is modified. In some aspects, the immune response is enhanced, stimulated or up-regulated by a bispecific protein. In some aspects, the immune response is inhibited or reduced by a bispecific protein. In some aspects, a bispecific protein exerts therapeutic effects without triggering a significant undesirable immune response, e.g., autoimmunity or inflammation.

In some aspects, a bispecific protein described herein is administered via a topical, epidermal mucosal, intranasal, oral, vaginal, rectal, sublingual, topical, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural or intrastemal route.

In some aspects, disclosed herein are methods of treating a disease or disorder a subject in need thereof, wherein the disease or disorder is an inflammatory disease or an autoimmune disease. Such subjects may exhibit an inadequate response to, or progressed on, a prior treatment, or have not previously received (i.e. been treated with) treatment for an inflammatory disease or an autoimmune disease. In some aspects, a bispecific protein disclosed herein is administered alone or with a standard of care treatment to treat an inflammatory disease or an autoimmune disease. In some aspects, a bispecific protein disclosed herein is administered as a maintenance therapy for an inflammatory disease or an autoimmune disease, e.g., a therapy that is intended to prevent the occurrence or recurrence of inflammation.

In some aspects, the inflammatory disease or autoimmune disease is selected from the group consisting of multiple sclerosis, rheumatoid arthritis, interstitial cystitis, type 1 diabetes, ulcerative colitis, Crohn's disease, psoriasis, psoriatic arthritis, ankylosing spondylitis, hidradenitis suppurativa, prurigo nodularis, non-infectious uveitis, renal/organ transplant, polymyalgia rheumatica, systemic lupus erythematous (SLE), cutaneous lupus erythematous, reduction of cardiovascular risk in CKD patients, gout, depression, inflammatory hand osteoarthritis, hand osteoarthritis, vitiligo, Graves' disease, asthma, non-eosinophilic asthma, non-eosinophilic COPD, alopecia areata, COVID-19 related CRS, calcium pyrophosphate deposition disease (psuedogout), CAR-T related CRS, interstitial lung disease (ILD), systemic sclerosis-associated interstitial lung disease (SSc-ILD), connective tissue disease-associated interstitial lung disease (CTD-ILD), Sjogren's syndrome, giant cell arteritis, systemic sclerosis (SSc), dermatomyositis/polymyositis, myasthenia gravis, antiphospholipid syndrome, sarcoidosis, lupus nephritis, IgG4-Related Disease(IgG4-RD), IgA nephropathy, immune thrombocytopeniaurpura (ITP), primary sclerosing cholangitis, primary biliary cirrhosis, focal segmental glomerulosclerosis (FSGS), multiple myeloma, pulmonary arterial hypertension, autoimmune hepatitis, Gougerot-Sjögren syndrome, macrophage activation syndrome, inclusion body myositis, multicentric Castleman's disease, cystoid macular edema, autoimmune myocarditis, pemphigus, bullous pemphigoid, chronic inflammatory demyelinating polyneuropathy, HCV-induced vasculitis, ANCA-associated vasculitis, Guillain Barre syndrome, multifocal motor neuropathy, anti-NMDAR encephalitis, acute graft versus host disease, chronic graft versus host disease, acute disseminated encephalomyelitis (ADEM), acute optic neuritis (AON), neuromyelitis optica (NMO), transverse myelitis, anti-MAG neuropathy, refractory aortitis, autoimmune hemolytic anemia, membranous nephropathy, Bechet's disease, Wegener's granulomatosis, Takayasu's disease, Kawasaki's disease, granulomatosis with polyangiitis, microscopic polyangiitis, Churg-Strauss syndrome, anti-glomerular basement membrane disease, hypocomplementemic urticarial vasculitis, IgA vasculitis (Henoch-Schönlein purpura), polyarteritis nodosa, Lichen planus, Schnitzler's syndrome, anti-MOG neuritis, thyroid eye disease, IL6 driven lymphoproliferative diseases (HLH, Langerhans), Adult-onset Still's disease (AOSD), juvenile idiopathic arthritis, relapsing polychondritis, chronic urticaria, eosinophilic asthma, food allergy desensitization, celiac disease, allergic rhinitis, allergic conjunctivitis, atopic dermatitis, eosinophilic esophagitis, eosinophilic COPD.

In some aspects, the disclosure relates to a method comprising administration of a bispecific protein disclosed herein for inducing an immune response in a subject with immunological deficiency syndromes. Immunological deficiency syndromes are diseases or conditions in which there is a loss of or defect in a component of the immune system. A bispecific protein can potentially restore or compensate the loss of an immune response in the subject to fight infectious diseases and cancer.

In some aspects, the disclosure relates to a method comprising administration of a bispecific protein disclosed herein for inducing or enhancing an immune response in a subject to treat infectious diseases. Examples of pathogens for which this therapeutic approach can be particularly useful, include pathogens for which there is currently no effective vaccine, or pathogens for which conventional vaccines are less than completely effective. These include, but are not limited to HIV, hepatitis (A, B, & C), influenza, herpes, giardia, malaria, leishmania, Staphylococcus aureus, Pseudomonas aeruginosa, and COVID-19.

In some aspects, the disclosure relates to a method comprising administration of a bispecific protein disclosed herein for inducing or enhancing an immune response in a subject to treat cancer. A bispecific protein can be used concurrently or sequentially with other cancer immunotherapies to treat patients with solid tumors or blood malignancies (liquid tumors). In some aspects, the cancer is a bladder cancer, breast cancer, uterine cancer, endometrial carcinoma, ovarian cancer, colorectal cancer, colon cancer, head and neck cancer, lung cancer, stomach cancer, germ cell cancer, bone cancer, squamous cell cancer, skin cancer, neoplasm of the central nervous system, lymphoma, leukemia, sarcoma, virus-related cancer, small-cell lung cancer, non-small cell lung cancer, gastrointestinal cancer, Hodgkin's or non-Hodgkin's lymphoma, pancreatic cancer, glioblastoma, glioma, cervical cancer, ovarian cancer, liver cancer, myeloma, salivary gland carcinoma, kidney cancer, basal cell carcinoma, melanoma, prostate cancer, vulval cancer, thyroid cancer, testicular cancer, esophageal cancer, or head or neck cancer, and any combinations thereof.

In some aspects, the disclosure relates to a method comprising administration of a bispecific protein disclosed herein with a vaccine for boosting an immune response to an antigen in a subject. Non-limiting examples of such antigens include tumor antigens or antigens from viruses, bacteria or other pathogens.

### EXAMPLES

The present technology is further illustrated by the following Examples, which should not be construed as limiting in any way. The examples herein are provided to illustrate advantages of the present technology and to further assist a person of ordinary skill in the art with preparing or using the compositions and systems of the present technology. The examples should in no way be construed as limiting the scope of the present technology, as defined by the appended claims.

### Example 1. α-IL6R/CTLA-4 Bispecific Proteins

Acting as a pro-inflammatory cytokine and growth factor, particularly for B cells, IL6 is a major player in chronic inflammatory diseases, autoimmune diseases, cancer and cytokine storm. Anti-IL6R agents have been approved by the FDA for treating immunological disorders, including rheumatoid arthritis (RA). However, there is a significant inter-individual variability of response to tocilizumab, an anti-IL6R monoclonal antibody. Only 30% patients display a remission, 20% of which do not respond anymore after 24 weeks. CD28, by engaging with CD80 and CD86, mediates the costimulatory signals that regulates the amplitude of T cell activation. CTLA-4 binds to the same ligands (CD80 and CD86) with a higher affinity than CD28. CTLA4-Ig has been developed as a competitive inhibitor of CD28 pathway and demonstrated efficacy in the clinic for treating RA. Since most immunological disorders are multifactorial in nature, involving the pathogenic inflammation, B cell, and T cell response, there is a strong rationale for the combination of IL6 and CD80/86 blockade. An α-IL6R/CTLA-4 bispecific protein disclosed herein can be administered to a subject to treat an inflammatory disease or an autoimmune disease.

The α-IL6R/CTLA-4 bispecific proteins disclosed herein have improved binding properties for each target. It is also designed to avoid the inherent shortcomings of CTLA-4-Ig. While blocking CD28, CTLA-4-Ig can also inhibit the signaling of endogenous CTLA-4, which is important for the function of Treg. Therefore, drugs in the CTLA4-Ig class, such as belatacept, can potentially inhibit Treg as well. This can not only compromise the efficacy of CTLA-4-Ig in treating autoimmune diseases but also increase the risk of developing post-transplant lymphoproliferative disease in certain patients. These issues could be avoided by certain aspects of the bispecific binding proteins disclosed herein. The designs can optimize target engagement between CD80/CD86 and the bispecific protein via forced proximity and multivalent binding involving IL6R as an anchor. These properties of an α-IL6R/CTLA-4 bispecific protein can translate into a higher target selectivity, a longer duration of action, and better efficacy in treating subjects with autoimmune diseases.

### Sequences and Constructs

In some aspects, the α-IL6R/CTLA-4 bispecific proteins were and can be created using the sequences identified in the Sequence Listing. The light chain of the molecule is derived from the light chain of an anti-IL6R antibody (SEQ ID NO: 1). The heavy chain of the molecule is selected from (SEQ ID NO: 3 to 10). The heavy chain is a fusion protein comprising the heavy chain of an anti-IL6R genetically fused via a flexible (Gly4Ser)ₙ linker to the N-terminus of the ectodomain of human CTLA-4 or its variants. Variant Fc regions may be used in some exemplary bispecific proteins to alter the effector function and/or half-life.

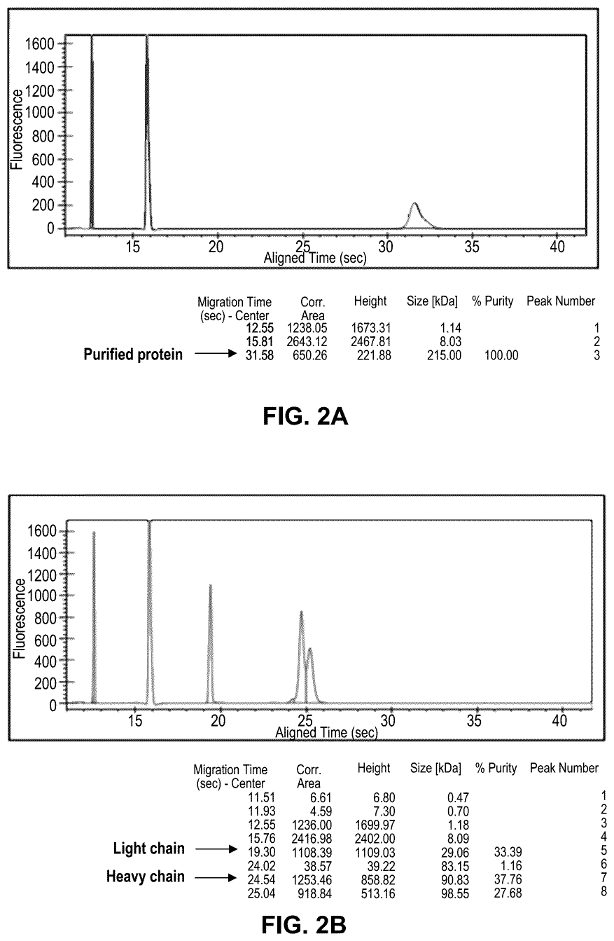

For expression of the bispecific proteins, the DNA encoding the light chain and DNA encoding the heavy chain fusion protein in either the same vector or separate vectors were used to transfect mammalian cells using standard protocols for transient or stable transfection. Conditioned culture media were harvested and the protein was purified by standard Protein A Sepharose chromatography. The purified protein (SEQ ID NO: 1 with SEQ ID NO: 3) has an estimated molecular weight (MW) of 215 kilodaltons by SDS-polyacrylamide electrophoresis (SDS-PAGE) under non-reducing conditions (FIG. 2A). Under reducing conditions, the light and heavy chains have apparent MW of 29 and 83-91 kilodaltons, respectively (FIG. 2B).

### Biacore Binding Assay

To evaluate binding of the bispecific binding proteins disclosed herein, surface plasmon resonance (SPR) studies were performed on a Biacore 8k instrument (GE Healthcare) at 25° C. The binding of the protein analytes was tested in buffer: 10 mM HEPES, 150 mM NaCl, 3 mM EDTA, 0.05% TWEEN20, pH7.4 on surfaces consisting of a low density (˜20RU) of human IL6R-Avi-tag or human CD80-Avi-tag which had been immobilized on NA-coated CM5 chips using standard protocol. The protein analytes were injected in a titration series at a flowrate of 50 ul/min. The data were analyzed using the Biacore Insight Evaluation software. 1:1 binding kinetic and affinity models are applied for experimental data analysis.

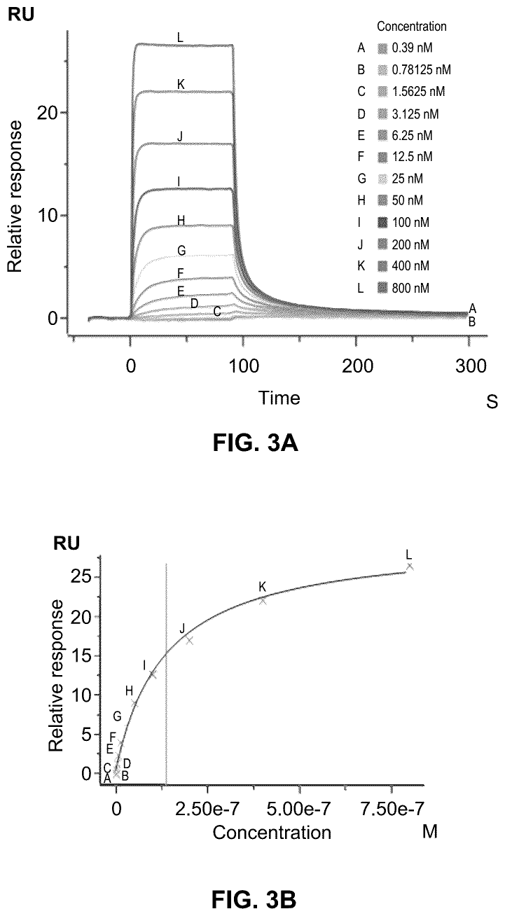

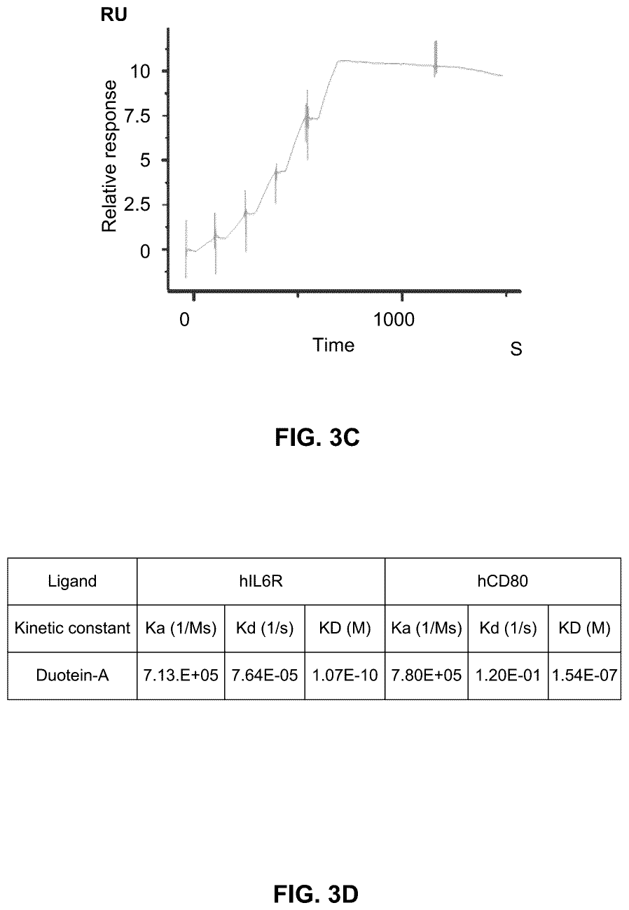

The binding results of an α-IL6R/CTLA-4 bispecific protein (SEQ ID NO: 1 with SEQ ID NO: 3), i.e., Duotein-A, are shown in FIGS. 3A-3D.

### Mixed Leukocyte Reaction (MLR) Assay

MLR assays were performed to test the impact α-IL6R/CTLA-4 bispecific proteins on T cell proliferation and activity.

CD14⁺ Monocytes were isolated from PBMC from healthy donors (EasySep monocyte enrichment kit, Stemcell). Monocytes were differentiated and matured into monocyte-derived dendritic cells (Mo-DC) in medium with GM-CSF/IL-4 (day 1-5), and were stimulated with 20 ug/mL LPS on day 6. Cells were immunophenotyped with CD14, CD80, CD86, HLA-DR, and CD83, and purity was confirmed to be >90%. Responder CD3+ T cells were prepared from a different donor using a negative selection kit (Stemcell). T cells were labeled with labeled with Celltrace-violet (Thermo Fisher). Cells were cocultured at a final ratio of T cells to Mo-DCs of 5:1. Various concentrations of α-IL6R/CTLA-4 bispecific protein, abatacept, or vehicle were added to the culture and incubated for 5 days. Cells were collected and analyzed by flow cytometry for T cell proliferation based on the dilution of Celltrace-violet. Supernatants were assessed for IL-2 and IFNγ by ELISA (Invitrogen).

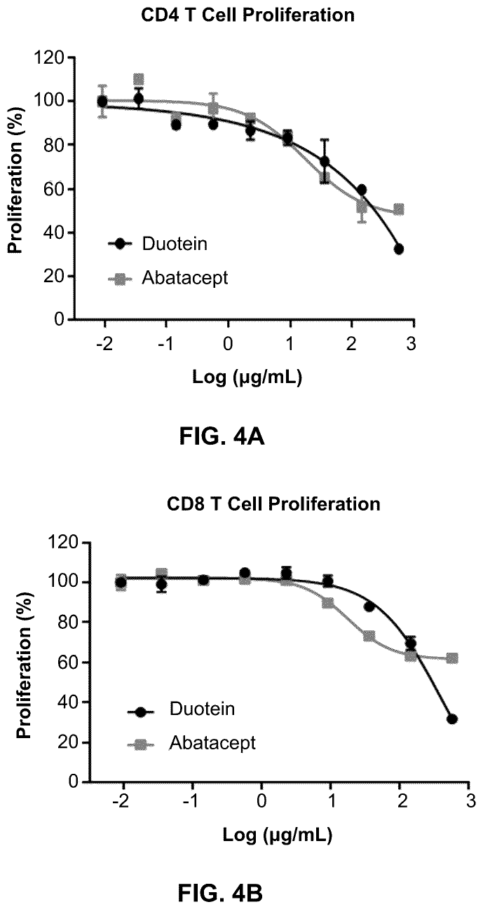

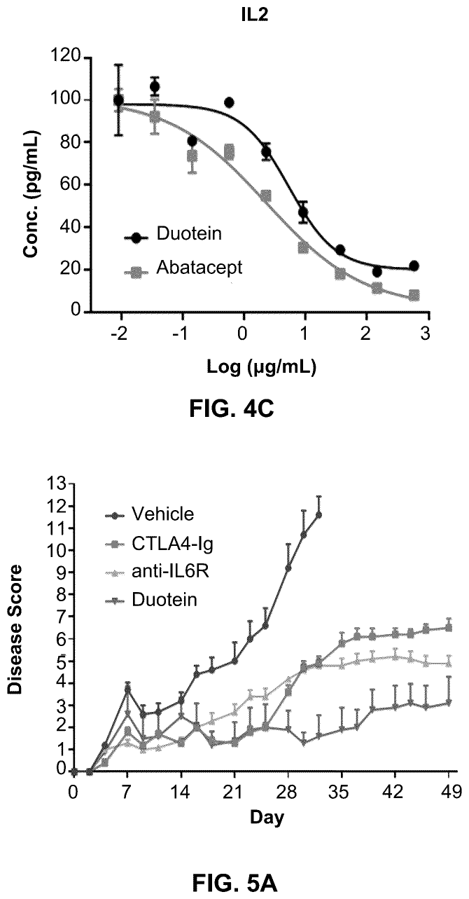

The results of one aspect in a MLR assay are shown in FIGS. 4A, 4B, and 4C. The α-IL6R/CTLA-4 bispecific protein (SEQ ID NO: 1 and NO: 3) was able to achieve a deeper inhibition of T cell proliferation than abatacept, a CTLA-4-Ig (FIGS. 4A and 4B). The activity of T cells, including secretion of IL2 and IFNγ, was also significantly suppressed by α-IL6R/CTLA-4 bispecific protein (FIG. 4C).

### Murine Model of Allogeneic Bone Marrow Transplant Induced-GvHD

Graft-versus-host disease (GVHD) is the major complication after allogeneic bone marrow transplantation and is characterized by the overproduction of proinflammatory cytokines and donor T cell-mediated tissue destruction. In this study, α-IL6R/CTLA-4 bispecific proteins were tested for its ability to prevent acute and chronic GvHD in a mouse model. A surrogate anti-IL6R that binds to murine IL6R was used to construct these α-IL6R/CTLA-4 bispecific proteins to be tested in the mouse GvHD model.

The mouse GvHD model involves the transfer of a mixture of bone marrow (BM) cells and splenocytes from a donor C57BL/6 strain to recipient BALB/c mice. Briefly, one day 0, BALB/c mice received 8 Gy total body irradiation. BM cell and splenocytes were collected from the C57BL/6 donors and suspended in PBS. Four hours after the irradiation, a BALB/c mouse was injected intravenously with 10 million BM cells and 5 million splenocytes as prepared. Recipients were monitored for food intake, weight loss, and clinical GvHD symptoms 3 times a week. Survival was recorded daily. Mice were treated with testing drugs or vehicle at 0.6 mg twice a week via i.p. injection from day 1 to day 40.

The severity of systemic GvHD developed in the mice was assessed according to a mouse clinical GvHD scoring system. The scoring system for acute GvHD had six clinical criteria with maximum index=11. Weight loss of <10% was scored 0, of >10% and <25% was scored as 1, and of >25% was scored as 2. For GI symptoms, the scoring system denoted 0 as normal and 1 as suffering from diarrhea. For posture and activity, the scoring system denoted 0 as normal, while 1 was used for hunching at rest and a mild to moderate decrease in activity, and 2 was used for severe hunching and a severe decrease in activity. For fur texture and skin integrity, the scoring system denoted 0 as normal, 1 for mild to moderate fur ruffling and scaling of the paws and tails, and 2 for severe fur ruffling and an obviously denuded mouse. Each mouse's total clinical GvHD score was measured 3 times a week.

The results of an α-IL6R/CTLA-4 bispecific protein are shown in FIGS. 5A and 5B. Administration of α-IL6R/CTLA-4 bispecific protein, i.e., Duotein, significantly reduced clinical severity of GvHD (FIG. 5A) and enhanced survival (FIG. 5B) when compared with vehicle, anti-IL6R or CTLA4-Ig treatment.

### Pharmacokinetics Study in Cynomolgus Monkeys

The pharmacokinetics was assessed in non-human primates, in which α-IL6R/CTLA-4 bispecific proteins have similar target binding affinity to that that in humans. Male cynomolgus monkeys were injected i.v. with 1 mg/kg or 10 mg/kg of an α-IL6R/CTLA-4 bispecific protein (n=3 each). Serum concentrations of the bispecific protein were determined from at various time points day 1 o day 14 following the standard protocol using a Gyrolab generic PK kit. Hematological parameters were assessed on day 14.

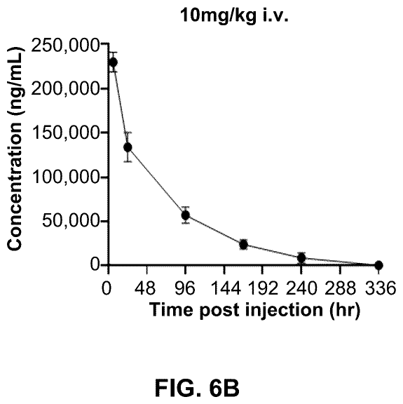

The results of an α-IL6R/CTLA-4 bispecific protein (SEQ ID NO: 1 and NO: 3) are shown in FIGS. 6A and 6B. The preliminary PK curves are characterized by a 2-exponential decay, with a target mediated disposition leading to rapid clearance within the first 24 hours post injection followed by a long elimination phase. The area under curve AUC and half-life increased non-proportionally when dose was increased to 10 mg/kg from 1 mg/kg, presumably due to a lower clearance after reaching a target saturation. The following parameters (predicted mean±SD) were estimated for a single dose of 10 mg/kg: Cₘₐₓ=275.6±14.7 μg/mL, AUCₗₐₛₜ=740±100 day*μg/mL, Vₛₛ=27.9±2.0 mL/kg, CL=16.7±1.9 mL/kg.

A 20% reduction of lymphocytes counts in the peripheral blood was observed on day 14 post administration of 10 mg/kg of an α-IL6R/CTLA-4 bispecific protein, while there were no significant changes in the counts of neutrophils and monocytes. This is consistent with the mechanism of activity by CTLA-4-Ig.

### Example 2. α-CD40/CTLA-4 Bispecific Proteins

CD40 is a tumor necrosis factor (TNF) receptor superfamily member and a costimulatory receptor constitutively expressed on antigen presenting cells. CD40 binds to its ligand CD40L expressed on activated T cells. The interaction of CD40 and CD40L activates a costimulatory signal, mediating a cross-talk between the adaptive and innate immune systems. The costimulatory signaling is important for geminal center function, antibody production, and humoral memory. It also regulates the function of macrophages and dendritic cells. The proinflammatory nature of CD40 signaling has been implicated in human autoimmune diseases, such as systemic lupus erythematosus, rheumatoid arthritis, and psoriatic arthritis. Monoclonal antibodies that block CD40 or CD40L have been tested in the clinic and demonstrated efficacy in treating autoimmune diseases and preventing rejection of transplant. Given the fact that both CD40 and CD80/86 are expressed on APCs and participate in the cross-talk between innate the adaptive immunity, we expect a synergy from a combination of CD40 and CD80/86 blockade. An α-CD40/CTLA-4 bispecific protein can improve target engagement via forced proximity compared to the mono-specific drugs for each target that are dosed separately. These properties of an α-CD40/CTLA-4 bispecific protein can translate into a higher target selectivity, a longer duration of action, and better efficacy in treating subjects with autoimmune diseases.

### Sequences and Constructs

In some embodiments, the α-CD40/CTLA-4 bispecific proteins were and can be created using the sequences identified in the SEQUENCE LISTINGS. The light chain of the molecule is derived from the light chain of an anti-CD40 antibody (SEQ ID NO: 2). The heavy chain of the molecule is selected from (SEQ ID NO: 11 to 18). The heavy chain is a fusion protein comprising the heavy chain of an anti-CD40 genetically fused via a flexible (Gly4Ser)ₙ linker to the N-terminus of the ectodomain of human CTLA-4 or its variants. Variant Fc regions may be used in some exemplary bispecific proteins to alter the effector function and/or half-life.

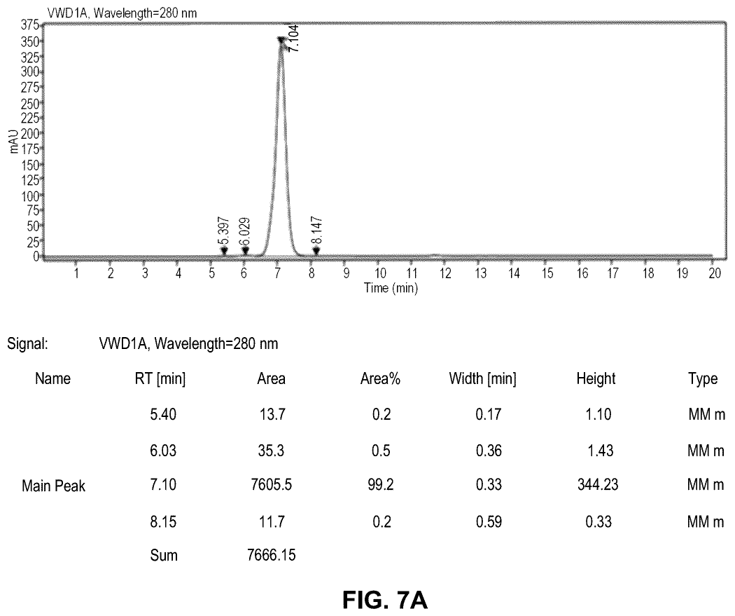

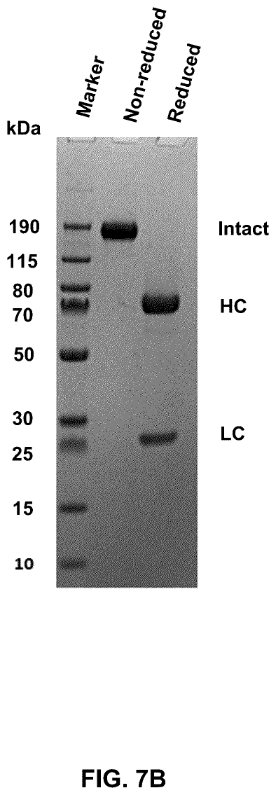

For expression of the bispecific proteins, the DNA encoding the light chain and DNA encoding the heavy chain fusion protein in either the same vector or separate vectors were used to transfect mammalian cells using standard protocols for transient or stable transfection. Conditioned culture media were harvested and the protein was purified by standard Protein A Sepharose chromatography. The purity of an exemplar α-CD40/CTLA-4 bispecific protein (SEQ ID NO: 2 with SEQ ID NO: 11) was assess by Size Exclusion Chromatography (HPLC-SEC) (FIG. 7A). The protein has an estimated molecular weight (MW) of about 200 kilodaltons by SDS-PAGE under non-reducing conditions. Under reducing conditions, the light and heavy chains have apparent MW of 25 and 75 kilodaltons, respectively (FIG. 7B).

### Biacore Binding Assay

To evaluate binding of the bispecific binding proteins disclosed herein, surface plasmon resonance (SPR) studies were performed on a Biacore 8k instrument (GE Healthcare) at 25° C. The binding of the protein analytes was tested in buffer: 10 mM HEPES, 150 mM NaCl, 3 mM EDTA, 0.05% TWEEN20, pH7.4 on surfaces consisting of a low density (˜200RU) of human CD40-Avi-tag or human CD80-Avi-tag which had been immobilized on NA-coated CM5 chips using standard protocol. The protein analytes were injected in a titration series at a flowrate of 50 ul/min. The data were analyzed using the Biacore Insight Evaluation software. An 1:1 binding kinetic and affinity models are applied for experimental data analysis. The value for the equilibrium dissociation constant (KD) was calculated as the ratio of the rate constants Kd/Ka.

The binding results of an α-CD40/CTLA-4 bispecific protein (SEQ ID NO: 2 with SEQ ID NO: 11), Duotein-B, are shown in FIGS. 8A-8C.

### Mixed Leukocyte Reaction (MIR) Assay

MLR assays were performed to test the impact of the α-CD40/CTLA-4 bispecific proteins on T cell proliferation and activity.

CD14⁺ Monocytes were isolated from PBMC from healthy donors (EasySep monocyte enrichment kit, Stemcell). Monocytes were differentiated and matured into monocyte-derived dendritic cells (Mo-DC) in medium with GM-CSF/IL-4 (day 1-5), and were stimulated with 20 ug/mL LPS on day 6. Cells were immunophenotyped with CD14, CD80, CD86, HLA-DR, and CD83, and purity was confirmed to be >90%. Responder CD3+ T cells were prepared from a different donor using a negative selection kit (Stemcell). T cells were labeled with labeled with Celltrace-violet (Thermo Fisher). Cells were cocultured at a final ratio of T cells to Mo-DCs of 5:1. Various concentrations of α-CD40/CTLA-4 bispecific protein, abatacept, or vehicle were added to the culture and incubated for 5 days. Cells were collected and analyzed by flowcytometry for T cell proliferation based on the dilution of Celltrace-violet. Supernatants were assessed for IL-2 and IFNγ by ELISA (Invitrogen).

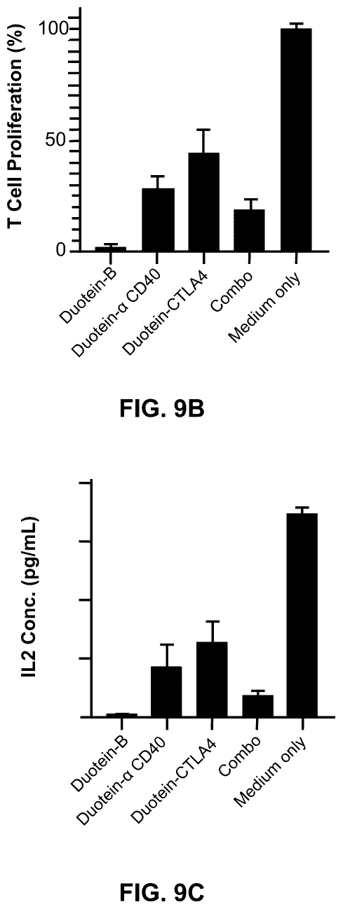

The results of an α-CD40/CTLA-4 bispecific protein in a MLR assay are shown in FIGS. 9A-9C. An exemplary α-CD40/CTLA-4 bispecific protein (SEQ ID NO: 2 and NO: 11), Duotein-B, was able to achieve a nearly complete inhibition of T cell proliferation. A control bispecific protein with the same CD80 binding motif but without the CD40-binding capability did not achieve such level of inhibition. Similarly, another control bispecific protein with the same CD40-binding motif but a suboptimal binding to CD80 failed to achieve such level of inhibition (FIG. 9A). Furthermore, a combination of the two control bispecific proteins underperformed in the same assay relative to the selected α-CD40/CTLA-4 bispecific protein (FIG. 9B). FIG. 9B shows Duotein-B achieved a more profound inhibition of T cell proliferation than a combination of two control bispecific proteins for single targets at an equal concentration of 100 μg/mL. The activity of T cells measured by secretion of IL2 was also significantly suppressed by an α-CD40/CTLA-4 bispecific protein (FIG. 9C). These data suggest a synergistic effect and superior features provided by the specific design of α-CD40/CTLA-4 bispecific protein, compared to the existing approaches of targeting each molecule separately or in combination.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A bispecific protein, comprising a first binding domain (BD1) that binds to a first target and a second binding domain (BD2) that binds to a second target (i) wherein the BD1 binds to IL6R, and BD2 binds to CD80/CD86; or (ii) wherein the BD1 binds to CD40 and BD2 binds to CD80/CD86.

2-20. (canceled)

21. The bispecific protein of claim 1, wherein the BD2 comprises an ectodomain of CTLA4.

22. The bispecific protein of claim 21, wherein the ectodomain of CTLA-4 comprises an amino acid sequence having at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to SEQ ID Nos: 35 to 40, wherein the amino acid sequence is capable of binding to CD80/CD86.

23. (canceled)

24. The bispecific protein of claim 1, wherein the BD1 comprises a heavy chain immunoglobulin variable domain (VH) and a light chain immunoglobulin variable domain (VL) that bind to IL6R, wherein:
(a) the VH comprises a VH-CDR1 sequence of SDHAWS (SEQ ID NO: 19), a VH-CDR2 sequence of YISYSGITTYNPSLKS (SEQ ID NO: 20), and a VH-CDR3 sequence of SLARTTAMDY (SEQ ID NO: 21), and
(b) the VL comprises a VL-CDR1 sequence of RASQDISSYLN (SEQ ID NO: 22), a VL-CDR2 sequence of YTSRLHS (SEQ ID NO: 23), and a VL-CDR3 sequence of QQGNTLPYT (SEQ ID NO: 24).

25. The bispecific protein of claim 24, wherein the VH comprises an amino acid sequence that has at least about 80%, at least about 85%, at least about 95%, at least about 96%, at least about 97%, at least about 98%, at least about 99%, or about 100% sequence identity to SEQ ID NO: 25; and/or the VL comprises an amino acid sequence that has at least about 80%, at least about 85%, at least about 95%, at least about 96%, at least about 97%, at least about 98%, at least about 99%, or about 100% sequence identity to SEQ ID NO: 26.

26-27. (canceled)

28. The bispecific protein of claim 1, wherein the BD1 comprises a heavy chain immunoglobulin variable domain (VH) and a light chain immunoglobulin variable domain (VL) derived from the following anti-IL6R antibodies: tocilizumab (SEQ ID NO: 42 and SEQ ID NO: 43), sarilumab (SEQ ID NO: 44 and SEQ ID NO: 45), satralizumab (SEQ ID NO: 46 and SEQ ID NO: 47), olokizumab (SEQ ID NO: 48 and SEQ ID NO: 49), and vobarilizumab (SEQ ID NO: 50).

29. (canceled)

30. The bispecific protein of claim 1, wherein the BD1 comprises a heavy chain immunoglobulin variable domain (VH) and a light chain immunoglobulin variable domain (VL), which bind to CD40, wherein:
(a) the VH comprises a VH-CDR1 sequence of GFTFSSYGMH (SEQ ID NO: 27), a VH-CDR2 sequence of VISYEESNRYHADSVKG (SEQ ID NO: 28), and a VH-CDR3 sequence of DGGIAAPGPDY (SEQ ID NO: 29), and
(b) the VL comprises a VL-CDR1 sequence of RSSQSLLYSNGYNYLD (SEQ ID NO: 30), a VL-CDR2 sequence of LGSNRAS (SEQ ID NO: 31), and a VL-CDR3 sequence of MQARQTPF (SEQ ID NO: 32).

31. The bispecific protein of claim 30, wherein the VH comprises an amino acid sequence that has at least about 80%, at least about 85%, at least about 95%, at least about 96%, at least about 97%, at least about 98%, at least about 99%, or about 100% sequence identity to SEQ ID NO: 33; and/or the VL comprises an amino acid sequence that has at least about 80%, at least about 85%, at least about 95%, at least about 96%, at least about 97%, at least about 98%, at least about 99%, or about 100% sequence identity to SEQ ID NO: 34.

32-33. (canceled)

34. The bispecific protein of claim 1, wherein the BD1 comprises a heavy chain immunoglobulin variable domain (VH) and a light chain immunoglobulin variable domain (VL) derived from the following anti-CD40 antibodies: iscalimab (SEQ ID NO: 51 and SEQ ID NO: 52), bleselumab (SEQ ID NO: 53 and SEQ ID NO: 54), ravagalimab (SEQ ID NO: 55 and SEQ ID NO: 56), lucatumumab (SEQ ID NO: 57 and SEQ ID NO: 58, BMS-986325 (SEQ ID NO: 59 and SEQ ID NO: 60), teneliximab (SEQ ID NO: 61 and SEQ ID NO: 62), BI-655064 (SEQ ID NO: 63 and SEQ ID NO: 64), and KPL-404 (SEQ ID NO: 65 and SEQ ID NO: 66).

35-41. (canceled)

42. A nucleic acid sequence encoding the bispecific protein of claim 1.

43. A vector comprising the nucleic acid molecule of claim 42.

44. A host cell comprising the vector of claim 43.

45-47. (canceled)

48. A pharmaceutical composition comprising the bispecific protein of claim 1 and a pharmaceutically acceptable excipient.

49. A kit comprising the bispecific protein of claim 1 and instructions for administering the bispecific protein to a subject in need thereof.

50. A method of producing a bispecific protein, comprising culturing the host cell of claim 44 under suitable conditions and recovering the bispecific protein.

51. A method of treating an immunological disorder in a subject in need thereof, comprising administering the bispecific protein of claim 1 to the subject.

52-55. (canceled)

56. A method of treating an infectious disease in a subject in need thereof, comprising administering the bispecific protein of claim 1 to the subject.

57. (canceled)

58. A method of treating cancer in a subject in need thereof, comprising administering the bispecific protein of claim 1 to the subject.

59. (canceled)

60. A method of boosting an immune response to an antigen in a subject, comprising administering the bispecific protein of claim 1 to the subject.

61-69. (canceled)


SEQUENCE LISTING (67 sequences)

SEQ ID NO: 1 - protein, 214 aa, synthetic construct [sequence omitted]

SEQ ID NO: 2 - protein, 219 aa, synthetic construct
  REGION 1..219: note = Peptide sequence of the light chain of an anti-CD40 antibody
DIVMTQSPLSLTVTPGEPASISCRSSQSLLYSNGYNYLDWYLQKPGQSPQVLISLGSNRASGVPDRFSGSGSGTDFTLKISRVEAEDVGVYYCMQARQTPFTFGPGTKVDIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 590 aa, synthetic construct
  REGION 1..590: note = Peptide sequence of the heavy chain of a Deutein: a-IL6R/CTLA-4 HC-1
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSGGGGSMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSD

SEQ ID NO: 4 - protein, 582 aa, synthetic construct
  REGION 1..582: note = Peptide sequence of the heavy chain of a Deutein: a-IL6R/CTLA-4 HC-2
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSGGGGSMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVID

SEQ ID NO: 5 - protein, 590 aa, synthetic construct
  REGION 1..590: note = Peptide sequence of the heavy chain of a Deutein: a-IL6R/CTLA-4 HC-3
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSGGGGSMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPAPDSD

SEQ ID NO: 6 - protein, 590 aa, synthetic construct
  REGION 1..590: note = Peptide sequence of the heavy chain of a Deutein: a-IL6R/CTLA-4 HC-4
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSGGGGSMHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSD

SEQ ID NO: 7 - protein, 582 aa, synthetic construct
  REGION 1..582: note = Peptide sequence of the heavy chain of a Deutein: a-IL6R/CTLA-4 HC-5
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSGGGGSMHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVID

SEQ ID NO: 8 - protein, 590 aa, synthetic construct
  REGION 1..590: note = Peptide sequence of the heavy chain of a Deutein: a-IL6R/CTLA-4 HC-6
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSGGGGSMHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPAPDSD

SEQ ID NO: 9 - protein, 593 aa, synthetic construct
  REGION 1..593: note = Peptide sequence of the heavy chain of a Deutein: a-IL6R/CTLA-4 HC-7
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSD

SEQ ID NO: 10 - protein, 593 aa, synthetic construct
  REGION 1..593: note = Peptide sequence of the heavy chain of a Deutein: a-IL6R/CTLA-4 HC-8
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSMHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSD

SEQ ID NO: 11 - protein, 591 aa, synthetic construct
  REGION 1..591: note = Peptide sequence of the heavy chain of a Deutein: a-CD40/CTLA-4 HC-1
QVQLVESGGGVVQPGRSLRLSCAASGFTFSSYGMHWVRQAPGKGLEWVAVISYEESNRYHADSVKGRFTISRDNSKITLYLQMNSLRTEDTAVYYCARDGGIAAPGPDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSGGGGSMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSD

SEQ ID NO: 12 - protein, 583 aa, synthetic construct
  REGION 1..583: note = Peptide sequence of the heavy chain of a Deutein: a-CD40/CTLA-4 HC-2
QVQLVESGGGVVQPGRSLRLSCAASGFTFSSYGMHWVRQAPGKGLEWVAVISYEESNRYHADSVKGRFTISRDNSKITLYLQMNSLRTEDTAVYYCARDGGIAAPGPDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSGGGGSMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVID

SEQ ID NO: 13 - protein, 591 aa, synthetic construct
  REGION 1..591: note = Peptide sequence of the heavy chain of a Deutein: a-CD40/CTLA-4 HC-3
QVQLVESGGGVVQPGRSLRLSCAASGFTFSSYGMHWVRQAPGKGLEWVAVISYEESNRYHADSVKGRFTISRDNSKITLYLQMNSLRTEDTAVYYCARDGGIAAPGPDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSGGGGSMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPAPDSD

SEQ ID NO: 14 - protein, 591 aa, synthetic construct
  REGION 1..591: note = Peptide sequence of the heavy chain of a Deutein: a-CD40/CTLA-4 HC-4
QVQLVESGGGVVQPGRSLRLSCAASGFTFSSYGMHWVRQAPGKGLEWVAVISYEESNRYHADSVKGRFTISRDNSKITLYLQMNSLRTEDTAVYYCARDGGIAAPGPDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSGGGGSMHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSD

SEQ ID NO: 15 - protein, 583 aa, synthetic construct
  REGION 1..583: note = Peptide sequence of the heavy chain of a Deutein: a-CD40/CTLA-4 HC-5
QVQLVESGGGVVQPGRSLRLSCAASGFTFSSYGMHWVRQAPGKGLEWVAVISYEESNRYHADSVKGRFTISRDNSKITLYLQMNSLRTEDTAVYYCARDGGIAAPGPDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSGGGGSMHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVID

SEQ ID NO: 16 - protein, 591 aa, synthetic construct
  REGION 1..591: note = Peptide sequence of the heavy chain of a Deutein: a-CD40/CTLA-4 HC-6
QVQLVESGGGVVQPGRSLRLSCAASGFTFSSYGMHWVRQAPGKGLEWVAVISYEESNRYHADSVKGRFTISRDNSKITLYLQMNSLRTEDTAVYYCARDGGIAAPGPDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSGGGGSMHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPAPDSD

SEQ ID NO: 17 - protein, 594 aa, synthetic construct
  REGION 1..594: note = Peptide sequence of the heavy chain of a Deutein: a-CD40/CTLA-4 HC-7
QVQLVESGGGVVQPGRSLRLSCAASGFTFSSYGMHWVRQAPGKGLEWVAVISYEESNRYHADSVKGRFTISRDNSKITLYLQMNSLRTEDTAVYYCARDGGIAAPGPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSD

SEQ ID NO: 18 - protein, 594 aa, synthetic construct
  REGION 1..594: note = Peptide sequence of the heavy chain of a Deutein: a-CD40/CTLA-4 HC-8
QVQLVESGGGVVQPGRSLRLSCAASGFTFSSYGMHWVRQAPGKGLEWVAVISYEESNRYHADSVKGRFTISRDNSKITLYLQMNSLRTEDTAVYYCARDGGIAAPGPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSMHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSD

SEQ ID NO: 19 - protein, 6 aa, synthetic construct
  REGION 1..6: note = Peptide sequence of acomplementarity-determining region of the heavy chain of an anti-IL6R antibody: a-IL6R VH-CDR1
SDHAWS

SEQ ID NO: 20 - protein, 16 aa, synthetic construct
  REGION 1..16: note = Peptide sequence of acomplementarity-determining region of the heavy chain of an anti-IL6R antibody: a-IL6R VH-CDR2
YISYSGITTYNPSLKS

SEQ ID NO: 21 - protein, 10 aa, synthetic construct
  REGION 1..10: note = Peptide sequence of acomplementarity-determining region of the heavy chain of an anti-IL6R antibody: a-IL6R VH-CDR3
SLARTTAMDY

SEQ ID NO: 22 - protein, 11 aa, synthetic construct
  REGION 1..11: note = Peptide sequence of a complementarity-determining region of the light chain of an anti-IL6R antibody: a-IL6R VL-CDR1
RASQDISSYLN

SEQ ID NO: 23 - protein, 7 aa, synthetic construct
  REGION 1..7: note = Peptide sequence of a complementarity-determining region of the light chain of an anti-IL6R antibody: a-IL6R VL-CDR2
YTSRLHS

SEQ ID NO: 24 - protein, 9 aa, synthetic construct
  REGION 1..9: note = Peptide sequence of a complementarity-determining region of the light chain of an anti-IL6R antibody: a-IL6R VL-CDR3
QQGNTLPYT

SEQ ID NO: 25 - protein, 119 aa, synthetic construct [sequence omitted]

SEQ ID NO: 26 - protein, 110 aa, synthetic construct
  REGION 1..110: note = Peptide sequence ofthelightchain variabledomain of an anti-IL6R antibody: a-IL6R VL
DIQMTQSPSSLSASVGDRVTITCRASQDISSYLNWYQQKPGKAPKLLIYYTSRLHSGVPSRFSGSGSGTDFTFTISSLQPEDIATYYCQQGNTLPYTFGQGTKVEIKRTV

SEQ ID NO: 27 - protein, 10 aa, synthetic construct
  REGION 1..10: note = Peptide sequence of acomplementarity-determining region of the heavy chain of an anti-CD40 antibody: a-CD40 VH-CDR1
GFTFSSYGMH

SEQ ID NO: 28 - protein, 17 aa, synthetic construct
  REGION 1..17: note = Peptide sequence of acomplementarity-determining region of the heavy chain of an anti-CD40 antibody: a-CD40 VH-CDR2
VISYEESNRYHADSVKG

SEQ ID NO: 29 - protein, 11 aa, synthetic construct
  REGION 1..11: note = Peptide sequence of acomplementarity-determining region of the heavy chain of an anti-CD40 antibody: a-CD40 VH-CDR3
DGGIAAPGPDY

SEQ ID NO: 30 - protein, 16 aa, synthetic construct
  REGION 1..16: note = Peptide sequence of a complementarity-determining region of the light chain of an anti-CD40 antibody: a-CD40 VL-CDR1
RSSQSLLYSNGYNYLD

SEQ ID NO: 31 - protein, 7 aa, synthetic construct
  REGION 1..7: note = Peptide sequence of a complementarity-determining region of the light chain of an anti-CD40 antibody: a-CD40 VL-CDR2
LGSNRAS

SEQ ID NO: 32 - protein, 9 aa, synthetic construct
  REGION 1..9: note = Peptide sequence of a complementarity-determining region of the light chain of an anti-CD40 antibody: a-CD40 VL-CDR3
MQARQTPFT

SEQ ID NO: 33 - protein, 120 aa, synthetic construct
  REGION 1..120: note = Peptide sequence of the heavy chain variable domain of an anti-CD40 antibody: a-CD40 VH
QVQLVESGGGVVQPGRSLRLSCAASGFTFSSYGMHWVRQAPGKGLEWVAVISYEESNRYHADSVKGRFTISRDNSKITLYLQMNSLRTEDTAVYYCARDGGIAAPGPDYWGQGTLVTVSS

SEQ ID NO: 34 - protein, 115 aa, synthetic construct
  REGION 1..115: note = Peptide sequence of the light chain variable domain of an anti-CD40 antibody: a-CD40 VL
DIVMTQSPLSLTVTPGEPASISCRSSQSLLYSNGYNYLDWYLQKPGQSPQVLISLGSNRASGVPDRFSGSGSGTDFTLKISRVEAEDVGVYYCMQARQTPFTFGPGTKVDIRRTV

SEQ ID NO: 35 - protein, 124 aa, synthetic construct [sequence omitted]

SEQ ID NO: 36 - protein, 116 aa, synthetic construct
  REGION 1..116: note = Peptide sequence of CTLA4 ectodomain variant-2
MHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVID

SEQ ID NO: 37 - protein, 124 aa, synthetic construct
  REGION 1..124: note = Peptide sequence of CTLA4 ectodomain variant-3
MHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPAPDSD

SEQ ID NO: 38 - protein, 124 aa, synthetic construct
  REGION 1..124: note = Peptide sequence of CTLA4 ectodomain variant-4
MHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSD

SEQ ID NO: 39 - protein, 116 aa, synthetic construct
  REGION 1..116: note = Peptide sequence of CTLA4 ectodomain variant-5
MHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVID

SEQ ID NO: 40 - protein, 124 aa, synthetic construct
  REGION 1..124: note = Peptide sequence of CTLA4 ectodomain variant-6
MHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPAPDSD

SEQ ID NO: 41 - protein, 6 aa, synthetic construct
MYPPPY

SEQ ID NO: 42 - protein, 119 aa, synthetic construct [sequence omitted]

SEQ ID NO: 43 - protein, 110 aa, synthetic construct; identical to SEQ ID NO: 26
  REGION 1..110: note = Peptide sequence of the light chain variable domain (VL) of tocilizumab

SEQ ID NO: 44 - protein, 116 aa, synthetic construct
  REGION 1..116: note = Peptide sequence of the heavy chain variable domain (VH) of sarilumab
EVQLVESGGGLVQPGRSLRLSCAASRFTFDDYAMHWVRQAPGKGLEWVSGISWNSGRIGYADSVKGRFTISRDNAENSLFLQMNGLRAEDTALYYCAKGRDSFDIWGQGTMVTVSS

SEQ ID NO: 45 - protein, 110 aa, synthetic construct
  REGION 1..110: note = Peptide sequence of the light chain variable domain (VL) of sarilumab
DIQMTQSPSSVSASVGDRVTITCRASQGISSWLAWYQQKPGKAPKLLIYGASSLESGVPSRFSGSGSGTDFTLTISSLQPEDFASYYCQQANSFPYTFGQGTKLEIKRTV

SEQ ID NO: 46 - protein, 119 aa, synthetic construct
  REGION 1..119: note = Peptide sequence of the heavy chain variable domain (VH) of satralizumab
QVQLQESGPGLVKPSETLSLTCAVSGHSISHDHAWSWVRQPPGEGLEWIGFISYSGITNYNPSLQGRVTISRDNSKNTLYLQMNSLRAEDTAVYYCARSLARTTAMDYWGEGTLVTVSS

SEQ ID NO: 47 - protein, 110 aa, synthetic construct
  REGION 1..110: note = Peptide sequence of the light chain variable domain (VL) of satralizumab
DIQMTQSPSSLSASVGDSVTITCQASTDISSHLNWYQQKPGKAPELLIYYGSHLLSGVPSRFSGSGSGTDFTFTISSLEAEDAATYYCGQGNRLPYTFGQGTKVEIERTV

SEQ ID NO: 48 - protein, 120 aa, synthetic construct [sequence omitted]

SEQ ID NO: 49 - protein, 110 aa, synthetic construct
  REGION 1..110: note = Peptide sequence of the light chain variable domain (VL) of olokizumab
DIQMTQSPSSLSASVGDRVTITCQADQDIGISLSWYQQKPGKAPKLLIYNANNLADGVPSRFSGSGSGTDFTLTISSLQPEDFATYYCLQHNSAPYTFGQGTKLEIKRTV

SEQ ID NO: 50 - protein, 121 aa, synthetic construct [sequence omitted]

SEQ ID NO: 51 - protein, 120 aa, synthetic construct; identical to SEQ ID NO: 33
  REGION 1..120: note = Peptide sequence of the heavy chain variable domain (VH) of iscalimab

SEQ ID NO: 52 - protein, 115 aa, synthetic construct; identical to SEQ ID NO: 34
  REGION 1..115: note = Peptide sequence of the light chain variable domain (VL) of iscalimab

SEQ ID NO: 53 - protein, 121 aa, synthetic construct
  REGION 1..121: note = Peptide sequence of the heavy chain variable domain (VH) of bleselumab
QLQLQESGPGLLKPSETLSLTCTVSGGSISSPGYYGGWIRQPPGKGLEWIGSIYKSGSTYHNPSLKSRVTISVDTSKNQFSLKLSSVTAADTAVYYCTRPVVRYFGWFDPWGQGTLVTVSS

SEQ ID NO: 54 - protein, 109 aa, synthetic construct
  REGION 1..109: note = Peptide sequence of the light chain variable domain (VL) of bleselumab
AIQLTQSPSSLSASVGDRVTITCRASQGISSALAWYQQKPGKAPKLLIYDASNLESGVPSRFSGSGSGTDFTLTISSLQPEDFATYYCQQFNSYPTFGQGTKVEIKRTV

SEQ ID NO: 55 - protein, 126 aa, synthetic construct
  REGION 1..126: note = Peptide sequence of the heavy chain variable domain (VH) of ravagalimab
HEAVYCHAINEVQLVESGGGLVKPGGSLRLSCAASGFTFSDYGMNWVRQAPGKGLEWIAYISSGRGNIYYADTVKGRFTISRDNAKNSLYLQMNSLRAEDTAVYYCARSWGYFDVWGQGTTVTVSS

SEQ ID NO: 56 - protein, 116 aa, synthetic construct
  REGION 1..116: note = Peptide sequence of the light chain variable domain (VL) of ravagalimab
DIVMTQSPDSLAVSLGERATINCKSSQSLLNRGNQKNYLTWFQQKPGQPPKLLIYWASTRESGVPDRFSGSGSGTDFTLTISSLQAEDVAVYYCQNDYTYPLTFGQGTKLEIKRTV

SEQ ID NO: 57 - protein, 120 aa, synthetic construct; identical to SEQ ID NO: 33
  REGION 1..120: note = Peptide sequence of the heavy chain variable domain (VH) of lucatumumab

SEQ ID NO: 58 - protein, 115 aa, synthetic construct; identical to SEQ ID NO: 34
  REGION 1..115: note = Peptide sequence of the light chain variable domain (VL) of lucatumumab

SEQ ID NO: 59 - protein, 117 aa, synthetic construct
  REGION 1..117: note = Peptide sequence of the heavy chain variable domain (VH) of BMS-986325
QVQLVQSGAEVKKPGSSVKVSCKASGYAFTSYWMHWVRQAPGQGLEWMGQINPTTGRSQYNEKFKTRVTITADKSTSTAYMELSSLRSEDTAVYYCARWGLQPFAYWGQGTLVTVSS

SEQ ID NO: 60 - protein, 107 aa, synthetic construct
  REGION 1..107: note = Peptide sequence of the light chain variable domain (VL) of BMS-986325
DIQMTQSPSFLSASVGDRVTITCKASQDVSTAVAWYQQKPGKAPKLLIYSASYRYTGVPSRFSGSGSGTDFTLTISSLQPEDFATYYCQQHYSTPWTFGGGTKVEIK

SEQ ID NO: 61 - protein, 122 aa, synthetic construct [sequence omitted]

SEQ ID NO: 62 - protein, 107 aa, synthetic construct [sequence omitted]

SEQ ID NO: 63 - protein, 119 aa, synthetic construct
  REGION 1..119: note = Peptide sequence of the heavy chain variable domain (VH) of BI-655064
EVQLVESGGGLVKPGGSLRLSCAASGFTFSDYGMHWVRQAPGKGLEWVAYISSGNRIIYYADTVKGRFTISRDNAKNSLYLQMNSLRAEDTALYYCARQDGYRYAMDYWAQGTLVTVSS

SEQ ID NO: 64 - protein, 116 aa, synthetic construct
  REGION 1..116: note = Peptide sequence of the light chain variable domain (VL) of BI-655064
DIVMTQSPDSLAVSLGERATMSCKSSQSLLNSGNQKNYLTWHQQKPGQPPKLLIYWTSTRESGVPDRFSGSGSGTDFTLTISSLQAEDVAVYYCQNDYTYPLTFGGGTKVEIKRTV

SEQ ID NO: 65 - protein, 113 aa, synthetic construct
  REGION 1..113: note = Peptide sequence of the heavy chain variable domain (VH) of KPL-404
QVQLVQSGAEKKPGASVKVSCKASGYTFTNYWMHWVRQAPGQRLEWMGYINPSNDYTKYNQKFKDRVTITRIDTSASTAYMELSSRSEDTAVYYCARQGFPYWGQGTLVTVSS

SEQ ID NO: 66 - protein, 106 aa, synthetic construct
  REGION 1..106: note = Peptide sequence of the light chain variable domain (VL) of KPL-404
EIVLTQSPATLSLSPGERATLSCSASSSVSYMHWYQQKPGQAPRLLIYDTSKLASGIPARFSGSGSGTDFTLTISSLEPEDFAVYYCHQLSSDPFTFGGGTKVEIK

SEQ ID NO: 67 - protein, 223 aa, synthetic construct
  REGION 1..223: note = Cytotoxic T-lymphocyte protein 4 (CTLA-4) or CD152
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN